(19) United States
(12) Patent Application Publication
(10) Pub. No.: US 2005/0260770 A1
(43) Pub. Date: Nov. 24, 2005

(54) Antigen array and diagnostic uses thereof

(76) Inventors: Irun R. Cohen, Rechovot (IL); Francisco Javier Quintana, Capital Federal (AR); Eytan Domany, Rechovot (IL); Gad Elizur, Jerusalem (IL); Peter H. Hagedorn, Copenhagen (DK)

Correspondence Address: Martin Moynihan;c/o ANTHONY CASTORINA, SUITE 207, 2001 JEFFERSON DAVIS HIGHWAY, ARLINGTON, VA, 22202, US

(21) Appl. No.: 11/094,142

(22) Filed: Mar. 31, 2005

Related U.S. Application Data
(60) Provisional application No. 60/558,137, filed on Apr. 1, 2004.

(51) Int. Cl. G01N 33/543

(52) U.S. Cl. 436/518

(57) ABSTRACT

A method of diagnosing an immune disease, or a predisposition thereto, in a subject is disclosed. The method comprises determining a capacity of immunoglobulins of the subject to specifically bind each antigen probe of an antigen probe set, wherein the antigen probe set comprises a plurality of antigen probes selected from the group consisting of at least a portion of a cell/tissue structure molecule, at least a portion of a heat shock protein, at least a portion of an immune system molecule, at least a portion of a homopolymeric polypeptide, at least a portion of a hormone, at least a portion of a metabolic enzyme, at least a portion of a microbial antigen, at least a portion of a molluscan antigen, at least a portion of a nucleic acid, at least a portion of a plant antigen, at least a portion of plasma molecule, and at least a portion of a tissue antigen, wherein the capacity is indicative of the immune disease or the predisposition thereto, thereby diagnosing the immune disease, or the predisposition thereto, in the subject.

This Patent Application claims priority from U.S. Provisional Patent Application No. 60/558,137, filed Apr. 1, 2004, the contents of which are hereby incorporated by reference.

### FIELD AND BACKGROUND OF THE INVENTION

The present invention relates to methods of diagnosing in a subject an immune disease, or predisposition thereto, to antigen probe arrays for practicing such a diagnosis, and to antigen probe sets for generating such arrays. More particularly, the present invention relates to methods of diagnosing in a subject an autoimmune disease, such as type 1 diabetes, or a predisposition thereto, to antigen probe arrays for practicing such a diagnosis, and to antigen probe sets for generating such arrays.

Immune diseases, such as autoimmune, transplantation-related, and allergic diseases, include numerous highly debilitating and/or lethal diseases whose medical management is suboptimal, for example, with respect to prevention, diagnosis, treatment, patient monitoring, prognosis, and/or drug design.

Autoimmune diseases represent a large group of highly debilitating and/or lethal diseases which includes such widespread and devastating diseases as type I diabetes, rheumatoid arthritis, inflammatory bowel disease, and multiple sclerosis.

Autoimmune diseases are marked by abundant autoantibodies and by vigorously responding T-cells targeted to selected self-antigens [Abbas, A. K., Lichtman, A. H. & Pober, J. S. (1994) Cellular and Molecular Immunology (W.B. Saunders Company, Philadelphia)]. Immunology has tended to focus on such blatant reactivities (Leslie, D. et al., 2001. J Clin Invest 108, 1417-22) and has paid relatively less attention to the autoimmunity detectable to non-classical self-antigens and to the low levels of global autoreactivity detected in healthy subjects (Martin, R. et al., 1990. J Immunol 145, 540-8; Moudgil, K. D. & Sercarz, E. E., 2000. Rev Immunogenet 2, 26-37; Lacroix-Desmazes, S. et al., 1998. J Immunol Methods 216, 117-37; Coutinho, A. et al., 1995. Curr Opin Immunol 7, 812-8).

Diabetes is broadly classified into two major types: the insulin dependent type (type 1 diabetes; juvenile diabetes) and the non insulin dependent type (type 2 diabetes; insulin resistant diabetes). Type 1 diabetes affects approximately 5 million people worldwide, and accounts for 5 to 10 percent of diagnosed diabetes in the United States. It is a devastating, life-long disease associated characterized by high mortality and morbidity (reviewed in The Diabetes Control and Complication Trial Research Group, 1993. N Engl J. Med. 329:977-986). Type 1 diabetes causes long-term complications which may affect virtually all parts of the body. In particular, diabetes frequently results in retinopathy leading to blindness, cardiovascular disease, stroke, nephropathy leading to kidney failure, and neuropathy (nerve damage), and may require amputation of affected body parts. Furthermore, diabetes may lead to complications during pregnancy, such as birth defects in babies born to women with the disease.

Diabetes is a disease caused by uncontrolled blood glucose levels as a result of a malfunction in the capacity of the body to metabolize glucose, its main source of metabolic energy, due to either lack or defect of insulin, a hormone secreted by the pancreas which uniquely functions to lower blood glucose levels. The pathogenesis of type I diabetes involves autoimmune attack and gradual elimination of the insulin secreting beta-cells of the pancreas. The consequence of the loss of beta-cells is a gradual shut-down of the insulin secreting capacity of the pancreas, and, as a result, sufferers of type 1 diabetes typically require daily insulin injections to survive. If not diagnosed and treated with insulin, a person with diabetes may lapse into a life-threatening diabetic coma, also known as diabetic ketoacidosis.

Type 1 diabetes occurs most often in young adults and children, however the condition can occur at any age. Symptoms of type 1 diabetes usually appear over a short period, however actual beta-cell depletion may begin years earlier. Presently, the causes of the body's immune rejection of its own beta-cells remain unknown, however genetic factors, and environmental factors, such as viruses, are thought to be involved. In humans, type 1 diabetes develops in persons bearing certain alleles, predominantly alleles of HLA immune response genes (Caillat-Zucman, S. & Bach, J. F., 2000. Clin Rev Allergy Immunol 19, 227-46), but most individuals who have inherited these susceptibility alleles will never develop the disease. Indeed, identical twins develop type 1 diabetes with a concordance rate of less than 50 percent, despite having inherited identical genomic DNA (Hyttinen, V. et al., 2003. Diabetes 52, 1052-5). Thus, environmental factors would appear to determine whether the diabetic potential inherent in one's genome becomes realized as type 1 diabetes [Bach, J. F., 2002. N Engl J Med 347, 911-20; Quintana, F. J. & Cohen, I. R.: Type 1 diabetes mellitus, infection and toll-like receptors. In Infection and Autoimmunity, Shoenfeld, Y. & Rose, N. (eds.), Elsevier, Amsterdam pp. 505-513 (2004)]. Since type 1 diabetes, including the CAD variant in NOD mice, is an autoimmune disease (Bach, J. F. & Mathis, D., 1997. Res Immunol 148, 285-6; Yasunami, R. & Bach, J. F., 1988. Eur J Immunol 18, 481-4; Tisch, R. & McDevitt, H., 1996. Cell 85, 291-7), it is very likely that resistance or susceptibility to the disease emerges from the interaction of the individual's immune system with the environment, down-stream of a permissive germ-line genetic endowment. Indeed, only the changing environment can be blamed for the alarming increase in the incidence of type 1 diabetes noted in the past few decades; significant changes in the frequencies of human genes have not occurred in the interim in affected populations (Bach, J. F., 1989. Contrib Microbiol Immunol 11, 263-88). Thus, type 1 diabetes emerges from the impact of the environment on the structure and function of the immune system in a way that transforms naturally benign autoimmunity into an autoimmune disease affecting the insulin-producing beta-cells. Various environmental factors can probably act to induce type 1 diabetes in susceptible individuals, as demonstrated in the cyclophosphamide-induced diabetes (CAD) model, since individual mice of the highly inbred NOD strain would seem to bear very similar, if not identical genomic DNA, yet almost half the male mice resist CAD as well as they resist slowly progressive spontaneous diabetes. Environmental factors can also prevent the development of type 1 diabetes. Stimulation of the NOD mouse immune system by infection (Oldstone, M. B., 1990. J Exp Med 171, 2077-89; Bras, A. & Aguas, A. P., 1996. Immunology 89, 20-5; Cooke, A. et al., 1999. Parasite Immunol 21, 169-76) or vaccination with microbial antigens (Elias, D. et al., 1991. Proc Natl Acad Sci USA 88, 3088-91) or by treatment with ligands that activate innate immune receptors (Serreze, D. V. et al., 1989. J Autoimmun 2, 759-76; Sai, P. & Rivereau, A. S., 1996. Diabetes Metab 22, 341-8; Quintana, F. J. et al., 2000. J. Immunol. 165, 6148-55) can prevent diabetes.

As mentioned hereinabove, immune diseases for which satisfactory diagnostic methods are lacking include transplantation-related and allergic diseases. Transplantation-related diseases such as graft rejection and graft-versus-host disease (GVHD) are major causes of failure of therapeutic transplantation, a medical procedure of last resort broadly practiced for treating numerous life-threatening diseases, such as cardiac, renal, pulmonary, hepatic and pancreatic failure. Allergic diseases, such as allergy to seasonal pollens, ragweed, dust mites, pet fur, cosmetics, and various common foods are significantly debilitating to a large proportion of the population, can be fatal, and are of great economic significance due to the large market for allergy drugs.

The humoral component of the adaptive immune system normally functions to afford rapid, specific and dynamic responses to a huge variety of antigen-specific insults in the form microbial pathogens and other foreign bodies. The antigenic specificity of humoral immune responses is determined via B-cell receptors (BCRs), antigen specific receptors clonally distributed on individual B-lymphocytes, whose repertoire of antigenic specificity is generated via somatic gene rearrangement. B-lymphocytes expressing BCRs capable of recognizing foreign antigens eventually secrete soluble BCRs, termed antibodies, which are the antigen-specific molecular effectors of humoral immunity. An antibody provides antigen-specific immunity by specifically binding to a cognate foreign antigen on the surface of a pathogen, such as virus, bacterium, or parasite, leading to their neutralization and elimination from the body via activation of the complement cascade culminating in oxidative burst killing of pathogen, and via Fc receptor mediated phagocytotic clearing of pathogen. The complement cascade further generates opsonins having the capacity to trigger non-specific inflammatory responses involving accumulation of phagocytes such as neutrophils and macrophages at sites of infection, thereby further sensitizing the immune system against the foreign antigen. While the function of humoral immune immunity is normally protective, failure of the regulatory mechanisms of such immunity to prevent attack against autoantigens results in autoimmune disease.

The need for optimal methods of diagnosing immune diseases, and predisposition thereto, is acutely felt in the pharmaceutical industry which depends on such methods for the development of new therapeutic biological agents and drugs. Immune diseases are intrinsically difficult to deal with pharmacologically. Not only are these diseases usually chronic, but the individual patients enrolled in treatment trials tend to be in different states of treatment-responsiveness. Thus it is difficult to devise a single dose of a drug and a treatment schedule that will be significant or optimal for each individual. Thus, even effective drugs have failed to demonstrate statistically significant therapeutic effects in clinical trials. Indeed, it is costly and hazardous to risk the success of a new drug on a long-term trial of one or a few doses or modes of administration. The industry critically requires relevant markers to stratify individuals according to their immunologic status so as to design trials geared towards achieving identification of optimal treatment regimens. Clinical trials of anti-inflammatory drugs have focused on the disease as the only endpoint, and have failed to monitor the cause of the disease. Hence, methods of characterizing antigenic specificities of the immune system could provide the information needed to optimize effectiveness and save time in arriving at dosing and other variables.

Traditionally, diagnosis of immune diseases, or predispositions thereto, has been based on an attempt to correlate each condition with a specific immune reactivity, such as an antibody or a T-lymphocyte response to a single antigen specific for the disease entity. This approach has been largely unsuccessful for various reasons, such as the absence of specific antigens serving as markers of the disease. In the case of autoimmune diseases, this approach has been unsuccessful due to, for example, immunity to multiple self-antigens, as exemplified by type I diabetes which may be associated with a dozen different antigens, and due to the fact that a significant number of healthy persons may manifest immune reactivities to self-antigens targeted in autoimmune diseases, such as insulin, DNA, myelin basic protein, thyroglobulin and others. For this reason, false positive tests are not uncommon. Hence, there is a real danger of making a false diagnosis based on the determination of a given immune reactivity.

Hence, there is an urgent need for improved methods of diagnosing immune diseases, such as type 1 diabetes, and predisposition thereto.

One potentially optimal strategy for diagnosing in a subject an immune disease, such as type 1 diabetes, or predisposition thereto, involves identification of global patterns of antigenic specificities of antibodies which generally correlate with such a disease or predisposition.

Various approaches for diagnosing in a subject an immune disease, such as type 1 diabetes, or predisposition thereto, via patterns of antigenic specificities of antibodies have been described in the prior art (reviewed in: Lieberman S M. and DiLorenzo T P., 2003. A comprehensive guide to antibody and T-cell responses in type 1 diabetes. Tissue Antigens. 62:359; Batstra M R. et al., 2001. Prediction and diagnosis of type 1 diabetes using beta-cell autoantibodies. Clin Lab. 7:497). For example, autoantibodies to HSP70 have been proposed to correlate with human type 1 diabetes (Abulafia-Lapid, R. et al., 2003. J Autoimmun. 20, 313-21), and coupled two-way clustering of human autoantibody reactivities has been proposed to have utility for separating human subjects already diabetic from healthy persons (Quintana, F. J. et al. 2003. J Autoimmun 21, 65-75). Another approach has attempted to identify patterns of disease-specific antigenic specificities of autoantibodies in subjects having established autoimmune diseases such as systemic lupus erythematosus (SLE) and rheumatoid arthritis (Robinson W H. et al., 2002. Nat Med. 8:295-301). A further approach has attempted to characterize the evolution of disease-specific antigenic specificities of autoantibodies during pathological progression of a murine autoimmune encephalitis model (Robinson W H. et al., 2003. Nat Biotechnol. 21:1033-9).

All of the aforementioned approaches, however, fail to provide optimal means of diagnosing immune diseases, such as type 1 diabetes, and in particular have failed to provide means of diagnosing predisposition to such diseases.

Thus, all prior art approaches have failed to provide an adequate solution for diagnosing occurrence, or predisposition, to autoimmune diseases such as diabetes.

There is thus a widely recognized need for, and it would be highly advantageous to have, a method devoid of the above limitation.

### SUMMARY OF THE INVENTION

The present invention discloses a method of diagnosing an immune disease, or predisposition thereto, in a subject. This method can be effected in a variety of ways as further described and exemplified hereinbelow.

According to one aspect of the present invention there is provided a method of diagnosing an immune disease, or a predisposition thereto, in a subject, the method comprising determining a capacity of immunoglobulins of the subject to specifically bind each antigen probe of an antigen probe set, wherein the antigen probe set comprises a plurality of antigen probes selected from the group consisting of at least a portion of a cell/tissue structure molecule, at least a portion of a heat shock protein, at least a portion of an immune system molecule, at least a portion of a homopolymeric polypeptide, at least a portion of a hormone, at least a portion of a metabolic enzyme, at least a portion of a microbial antigen, at least a portion of a molluscan antigen, at least a portion of a nucleic acid, at least a portion of a plant antigen, at least a portion of plasma molecule, and at least a portion of a tissue antigen, wherein the capacity is indicative of the immune disease or the predisposition thereto, thereby diagnosing the immune disease, or the predisposition thereto, in the subject.

According to further features in preferred embodiments of the invention described below, the immunoglobulins belong to the IgG isotype, and the plurality of antigen probes is selected from the group consisting of at least a portion of a human 70 kDa heat shock protein, at least a portion of a cardiac hormone, at least a portion of a neurotransmitter, at least a portion of a pigmentation hormone, at least a portion of a vascular hormone, at least a portion of an albumin, at least a portion of a lipoprotein, at least a portion of a nerve system molecule, and at least a portion of a pancreatic molecule.

According to still further features in the described preferred embodiments, the immunoglobulins belong to the IgG isotype, and the plurality of antigen probes is selected from the group consisting of a bacterial heat shock protein, at least a portion of a 60 kDa human heat shock protein, at least a portion of a 70 kDa human heat shock protein, at least a portion of a metabolic enzyme, at least a portion of a lipopolysaccharide, at least a portion of a molluscan antigen, at least a portion of a wheat antigen, and at least a portion of a nerve system molecule.

According to still further features in the described preferred embodiments, the immunoglobulins belong to the IgG isotype, and the plurality of antigen probes is selected from the group consisting of at least a portion of a 60 kDa human heat shock protein, at least a portion of a 70 kDa human heat shock protein, at least a portion of a cytokine receptor, at least a portion of a T-cell receptor, at least a portion of a metabolic enzyme, at least a portion of a coagulation regulator, at least a portion of a nerve system molecule, and at least a portion of a pancreatic molecule.

According to still further features in the described preferred embodiments, the immunoglobulins belong to the IgG isotype, and the plurality of antigen probes is selected from the group consisting of at least a portion of a bacterial heat shock protein, at least a portion of a human 60 kDa heat shock protein, at least a portion of a human 70 kDa heat shock protein, and at least a portion of a glutathione S-transferase.

According to still further features in the described preferred embodiments, the immunoglobulins belong to the IgM isotype, and the plurality of antigen probes is selected from the group consisting of at least a portion of a muscle structure protein, at least a portion of a 70 kDa human heat shock protein, at least a portion of a homopolymeric polypeptide, at least a portion of a hypothalamic hormone, at least a portion of a neurotransmitter, at least a portion of a matrix metalloproteinase, at least a portion of a viral antigen, at least a portion of a wheat antigen, at least a portion of an albumin, and at least a portion of a lipoprotein.

According to still further features in the described preferred embodiments, the immunoglobulins belong to the IgM isotype, and the plurality of antigen probes is selected from the group consisting of at least a portion of a crystallin, at least a portion of a bacterial heat shock protein, at least a portion of a human 60 kDa heat shock protein, at least a portion of a human 70 kDa heat shock protein, at least a portion of a complement molecule, at least a portion of a cytokine, and at least a portion of a nucleic acid.

According to still further features in the described preferred embodiments, the immunoglobulins belong to the IgM isotype, and the plurality of antigen probes is selected from the group consisting of at least a portion of a human 70 kDa heat shock protein, at least a portion of a cytokine, at least a portion of a metabolic enzyme, at least a portion of a viral antigen, at least a portion of a molluscan antigen, at least a portion of an albumin, at least a portion of a lipoprotein, at least a portion of a cartilage antigen, and at least a portion of a nerve system molecule.

According to still further features in the described preferred embodiments, the immunoglobulins belong to the IgM isotype, and the plurality of antigen probes is selected from the group consisting of at least a portion of a bacterial heat shock protein, at least a portion of a human 60 kDa heat shock protein, at least a portion of a human 70 kDa heat shock protein, at least a portion of T-cell receptor, at least a portion of a lipopolysaccharide and at least a portion of a pancreatic molecule.

According to still further features in the described preferred embodiments, determining the capacity of the immunoglobulins to specifically bind each antigen probe of the plurality of antigen probes comprises: (a) exposing the immunoglobulins to the plurality of antigen probes, wherein each antigen probe of the plurality of antigen probes is attached to a distinct addressable location of a plurality of addressable locations of a support of an antigen probe array; and (b) measuring a hybridization of the each antigen probe of the plurality of antigen probes with the immunoglobulins.

According to still further features in the described preferred embodiments, the immune disease is an autoimmune disease.

According to still further features in the described preferred embodiments, the immune disease is type I diabetes.

According to still further features in the described preferred embodiments, the immune disease is a pancreatic immune disease.

According to another aspect of the present invention there is provided an antigen probe array comprising: (a) a support which comprises a plurality of addressable locations; and (b) an antigen probe set which comprises a plurality of antigen probes selected from the group consisting of at least a portion of a cell/tissue structure molecule, at least a portion of a heat shock protein, at least a portion of an immune system molecule, at least a portion of a homopolymeric polypeptide, at least a portion of a hormone, at least a portion of a metabolic enzyme, at least a portion of a microbial antigen, at least a portion of a molluscan antigen, at least a portion of a nucleic acid, at least a portion of a plant antigen, at least a portion of plasma molecule, and at least a portion of a tissue antigen, wherein each antigen probe of the plurality of antigen probes is attached to a specific addressable location of the plurality of addressable locations.

According to yet another aspect of the present invention there is provided an antigen probe set comprising a plurality of antigen probes selected from the group consisting of at least a portion of a cell/tissue structure molecule, at least a portion of a heat shock protein, at least a portion of an immune system molecule, at least a portion of a homopolymeric polypeptide, at least a portion of a hormone, at least a portion of a metabolic enzyme, at least a portion of a microbial antigen, at least a portion of a molluscan antigen, at least a portion of a nucleic acid, at least a portion of a plant antigen, at least a portion of plasma molecule, and at least a portion of a tissue antigen.

According to further features in preferred embodiments of the invention described below, the plurality of antigen probes is selected from the group consisting of at least a portion of a human 70 kDa heat shock protein, at least a portion of a cardiac hormone, at least a portion of a neurotransmitter, at least a portion of a pigmentation hormone, at least a portion of a vascular hormone, at least a portion of an albumin, at least a portion of a lipoprotein, at least a portion of a nerve system molecule, and at least a portion of a pancreatic molecule.

According to still further features in the described preferred embodiments, the plurality of antigen probes is selected from the group consisting of a bacterial heat shock protein, at least a portion of a 60 kDa human heat shock protein, at least a portion of a 70 kDa human heat shock protein, at least a portion of a metabolic enzyme, at least a portion of a lipopolysaccharide, at least a portion of a molluscan antigen, at least a portion of a wheat antigen, and at least a portion of a nerve system molecule.

According to still further features in the described preferred embodiments, the plurality of antigen probes is selected from the group consisting of at least a portion of a 60 kDa human heat shock protein, at least a portion of a 70 kDa human heat shock protein, at least a portion of a cytokine receptor, at least a portion of a T-cell receptor, at least a portion of a metabolic enzyme, at least a portion of a coagulation regulator, at least a portion of a nerve system molecule, and at least a portion of a pancreatic molecule.

According to still further features in the described preferred embodiments, the plurality of antigen probes is selected from the group consisting of at least a portion of a bacterial heat shock protein, at least a portion of a human 60 kDa heat shock protein, at least a portion of a human 70 kDa heat shock protein, and at least a portion of a glutathione S-transferase.

According to still further features in the described preferred embodiments, the plurality of antigen probes is selected from the group consisting of at least a portion of a muscle structure protein, at least a portion of a 70 kDa human heat shock protein, at least a portion of a homopolymeric polypeptide, at least a portion of a hypothalamic hormone, at least a portion of a neurotransmitter, at least a portion of a matrix metalloproteinase, at least a portion of a viral antigen, at least a portion of a wheat antigen, at least a portion of an albumin, and at least a portion of a lipoprotein.

According to still further features in the described preferred embodiments, the plurality of antigen probes is selected from the group consisting of at least a portion of a crystallin, at least a portion of a bacterial heat shock protein, at least a portion of a human 60 kDa heat shock protein, at least a portion of a human 70 kDa heat shock protein, at least a portion of a complement molecule, at least a portion of a cytokine, and at least a portion of a nucleic acid.

According to still further features in the described preferred embodiments, the plurality of antigen probes is selected from the group consisting of at least a portion of a human 70 kDa heat shock protein, at least a portion of a cytokine, at least a portion of a metabolic enzyme, at least a portion of a viral antigen, at least a portion of a molluscan antigen, at least a portion of an albumin, at least a portion of a lipoprotein, at least a portion of a cartilage antigen, and at least a portion of a nerve system molecule.

According to still further features in the described preferred embodiments, the plurality of antigen probes is selected from the group consisting of at least a portion of a bacterial heat shock protein, at least a portion of a human 60 kDa heat shock protein, at least a portion of a human 70 kDa heat shock protein, at least a portion of T-cell receptor, at least a portion of a lipopolysaccharide and at least a portion of a pancreatic molecule.

The present invention successfully addresses the shortcomings of the presently known configurations by identifying for the first time, and by providing a means of determining, global patterns of antigenic specificities of antibodies of a subject which effectively correlate with occurrence or predisposition of an immune disease such as type 1 diabetes, in the subject.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a flowchart depicting the experimental protocol as a function of time. The numbers refer to the age (in weeks) of the mice. The black vertical lines at 4 and 9 weeks indicate serum sample collection. The grey vertical lines at 4 and 5 weeks indicate cyclophosphamide injection. The grey box at 6 weeks shows when the CAD-susceptible mice developed diabetes, and the grey box between 11 and 13 weeks shows the time of death of the untreated diabetic mice.

FIG. 2 is a diagram depicting reactivity matrices of 27 antigens that separate diabetic and healthy mice. The rows are antigens and the columns are the mouse sera. Each antigen is identified by the number between the two reactivity matrices. See Table 2, antigen ID no.'s, for antigen identification. The left panel shows a two-way SPC of the antigens and the SB and HB samples. The length of a branch connecting to a cluster represents the stability of the cluster. Filled boxes denote mice that later developed CAD, and open boxes denote mice that resisted CAD. The right panel shows the SPC of the SA and HA samples post-CAD. Filled boxes denote mice that developed CAD, and open boxes denote healthy mice that resisted CAD. The antigens used in the two panels are the same and presented in the same order.

FIG. 3 is a diagram depicting two-way SPC of the 27 antigens (numbered) that best separate SA and HA mice, identified applying the Wilcoxon rank-sum test to the reactivities. Filled boxes denote diabetic mice and open boxes denote healthy mice.

FIG. 4 is a diagram depicting two-way SPC of the 27 antigens (numbered) that best separate the sick and healthy mouse samples, identified applying the Wilcoxon rank-sum test to the pre-CAD and post-CAD ratios. Filled boxes denote mice susceptible to CAD and open boxes denote mice resistant to CAD.

FIG. 5 is a Venn diagram showing antigens that were unique to each set or shared by any of the three lists of 27 antigens that best separated the groups of mice: (list I), SB and HB; (list II), SA and HA; and (list III), sick and healthy by ratio. See Table 2, antigen ID no.'s, for antigen identification.

FIG. 6 is a diagram depicting reactivity matrices of IgM-reactive or IgG-reactive antigens that separate diabetic and healthy mice.

FIG. 7 is a schematic diagram depicting an antigen probe array.

### DESCRIPTION OF THE PREFERRED EMBODIMENTS

The present invention is of methods of diagnosing in a subject an immune disease, or a predisposition thereto; of antigen probe arrays for performing such diagnosis, and of antigen probe sets for generating such arrays. Specifically, the present invention can be used to characterize in a subject a global pattern of antigenic specificities of antibodies correlating with an existant immune disease such as type 1 diabetes, or predisposition thereto. As such the present invention can be used to diagnose with enhanced effectiveness relative to the prior art an immune disease such as type 1 diabetes, or a predisposition thereto, in a subject. Therefore, the methods, probe arrays and probe sets of the present invention enable improved prophylactic and therapeutic treatment of immune diseases such as type 1 diabetes.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

No optimal methods are available for medical management (e.g., prevention, diagnosis, treatment, patient monitoring, prognosis, drug design, and the like) of numerous lethal/debilitating immune diseases, such as autoimmune, transplantation-related, and allergic diseases. An optimal strategy for facilitating medical management of such a disease in an individual would be via a method enabling improved diagnosis of the disease or predisposition thereto in the individual.

Various methods of diagnosing in a subject an immune disease, or predisposition thereto, via antibody antigenic specificity patterns have been described by the prior art (reviewed in: Lieberman S M. and DiLorenzo T P., 2003. A comprehensive guide to antibody and T-cell responses in type 1 diabetes. Tissue Antigens. 62:359; Batstra M R. et al., 2001. Prediction and diagnosis of type 1 diabetes using beta-cell autoantibodies. Clin Lab. 7:497). For example, autoantibodies to HSP70 have been proposed to correlate with human type 1 diabetes (Abulafia-Lapid, R. et al., 2003. J Autoimmun 20, 313-21), and coupled two-way clustering of human autoantibody reactivities has been proposed to have utility for separating human subjects already diabetic from healthy persons (Quintana, F. J. et al. 2003. J Autoimmun 21, 65-75). Further approaches has attempted to identify patterns of disease-specific antigenic specificities of autoantibodies in subjects having established autoimmune diseases such as systemic lupus erythematosus (SLE) and rheumatoid arthritis (Robinson W H. et al., 2002. Nat Med. 8:295-301), and to characterize the evolution of disease-specific antigenic specificities of autoantibodies during pathological progression of a murine autoimmune encephalitis model (Robinson W H. et al., 2003. Nat Biotechnol. 21:1033-9).

However, all such prior art approaches fail to provide optimal means of correlating patterns of antigenic specificity of antibodies of a subject with occurrence of an immune disease, such as type 1 diabetes, and in particular fail to provide means of diagnosing predisposition to such a disease.

Thus, the prior art fails to enable optimal diagnosis of immune diseases such as type 1 diabetes, or predisposition thereto, and thereby fails to enable optimal medical management of such diseases.

While reducing the present invention to practice global patterns of antigenic specificity of antibodies correlating with predisposition to, or onset of, type 1 diabetes in test subjects were uncovered for the first time.

Hence, the present invention enables dramatically improved correlation of patterns of antigenic specificities of antibodies with onset of, or predisposition to, an immune disease, such as type 1 diabetes, relative to the prior art. As such, the present invention enables optimal diagnosis of an immune disease such as type 1 diabetes, or predisposition thereto, relative to the prior, and thereby enables optimal medical management of such a disease relative to the prior art.

Thus, according to the present invention, there is provided a method of diagnosing an immune disease, or a predisposition thereto, in a subject. The method is effected by determining the capacity of immunoglobulins of the subject to specifically bind each antigen probe of an antigen probe set where such capacity is indicative of the immune disease or the predisposition thereto.

According to the teachings of the present invention, the probe set comprises a plurality of antigen probes selected from the group consisting of at least a portion of a celutissue structure molecule, at least a portion of a heat shock protein, at least a portion of an immune system molecule, at least a portion of a homopolymeric polypeptide, at least a portion of a hormone, at least a portion of a metabolic enzyme, at least a portion of a microbial antigen, at least a portion of a molluscan antigen, at least a portion of a nucleic acid, at least a portion of a plant antigen, at least a portion of a plasma molecule, and at least a portion of a tissue antigen.

As used herein, the phrase “immune disease” refers to any disease associated with a pathogenic or protective antigen specific immune response. Specific examples of immune diseases, and predispositions thereto, which can be diagnosed via the method of the present invention are described hereinbelow.

The present invention is particularly useful for diagnosing an immune disease such as type 1 diabetes, or a predisposition thereto, in a mammalian subject such as a rodent or a primate. Preferably, the rodent is a mouse. The primate is preferably a human.

As used herein, the term “disease” refers to any medical disease, disorder, condition, or syndrome, or to any undesired and/or abnormal physiological morphological, and/or physical state and/or condition.

As further described hereinbelow, the method of the present invention may be effected in any of various ways, depending on the application and purpose, including via use of an antigen probe set which may include any of various combinations of antigen probes of the present invention, and by determining in any of various ways the capacity of immunoglobulins of the subject to specifically bind the antigen probes.

A preferred cell/tissue structure molecule is a muscle structure protein.

A preferred muscle structure protein is troponin. A suitable troponin is rabbit muscle troponin.

Preferred heat shock proteins are crystallin molecules, bacterial heat shock proteins, and human heat shock proteins.

A preferred crystallin molecule is alpha-crystallin. A suitable alpha crystallin molecule is bovine alpha-crystallin.

Preferred bacterial heat shock proteins are E. coli GroEL (65 kDa heat shock protein) and Mycobacterium tuberculosis 71 kDa heat shock protein (HSP71).

Where the heat shock protein is GroEL, a preferred at least a portion of the heat shock protein is a polypeptide which comprises at least one amino acid sequence selected from those set forth by SEQ ID NOs: 1, 2, 3, 4, 5, 6, 7, 8, 9, 10, 11 and 12. Preferred polypeptides which comprises at least one amino acid sequence selected from those set forth by SEQ ID NOs: 1, 2, 3, 4, 5, 6, 7, 8, 9, 10, 11 and 12, are the 12 polypeptides having the amino acid sequences set forth by SEQ ID NOs: 1, 2, 3, 4, 5, 6, 7, 8, 9, 10, 11 and 12, respectively.

Preferred human heat shock proteins are human 60 kDa heat shock protein (HSP60), and human 70 kDa heat shock protein (HSP70).

Where the heat shock protein is HSP60, a preferred at least a portion of the heat shock protein is a polypeptide which comprises at least one amino acid sequence selected from those set forth by SEQ ID NOs: 13, 14, 15, 16, 17, 18, 19 and 20. Preferred polypeptides which comprise at least one amino acid sequence selected from those set forth by SEQ ID NOs: 13, 14, 15, 16, 17, 18, 19 and 20 are the eight polypeptides having the amino acid sequences set forth by SEQ ID NOs: 13, 14, 15, 16, 17, 18, 19 and 20, respectively.

Where the heat shock protein is HSP70, a preferred at least a portion of the heat shock protein is a polypeptide which comprises at least one amino acid sequence selected from those set forth by SEQ ID NOs: 21, 22, 23, 24, 25, 26, 27, 28, 29, 30, 31, 32, 33, 34, 35, 36, 37, 38 and 39. Preferred polypeptides which comprise at least one amino acid sequence selected from those set forth by SEQ ID NOs: 21, 22, 23, 24, 25, 26, 27, 28, 29, 30, 31, 32, 33, 34, 35, 36, 37, 38 and 39 are the 19 polypeptides having the amino acid sequences set forth by SEQ ID NOs: 21, 22, 23, 24, 25, 26, 27, 28, 29, 30, 31, 32, 33, 34, 35, 36, 37, 38 and 39, respectively.

Preferred immune system molecules are complement molecules, cytokines, cytokine receptors, and T-cell receptors (TCRs).

A preferred complement molecule is complement C5. A preferred complement C5 molecule is human complement C5.

Preferred cytokines are interleukins and tumor necrosis factors (TNFs).

Preferred interleukins are interleukin-4 (IL-4) and interleukin-10 (IL-10).

A preferred interleukin-4 is of the same species as the subject.

A preferred interleukin-10 is of the same species as the subject.

A preferred tumor necrosis factor is tumor necrosis factor-alpha (TNF-alpha).

A preferred cytokine receptor is interleukin-2 receptor (IL-2R).

Where the immune system molecule is interleukin-2 receptor, a preferred at least a portion of an immune system molecule is a polypeptide which comprises at least one amino acid sequence selected from those set forth by SEQ ID NOs: 40 and 41. Preferred polypeptides which comprise at least one amino acid sequence selected from those set forth by SEQ ID NOs: 40 and 41 are the two polypeptides having the amino acid sequences set forth by SEQ ID NO: 40 and SEQ ID NO: 41, respectively.

Where the immune system molecule is a T-cell receptor, a preferred at least a portion of an immune system molecule is a T-cell receptor beta-chain or a polypeptide which comprises the amino acid sequence set forth by SEQ ID NO: 42. A suitable TCR beta-chain is rat T-cell receptor beta-chain C2 (described in Jewtoukoff V. et al., 1992. Scand J. Immunol. 36:893). A preferred polypeptide which comprises the amino acid sequence set forth by SEQ ID NO: 42 is a polypeptide having the amino acid sequence set forth by SEQ ID NO: 42.

Preferred homopolymeric polypeptides are poly-L-aspartic acid [poly(Asp)] and homopolymeric polypeptides having a molecular weight selected from a range of about 15 kDa to about 50 kDa. A most preferred homopolymeric polypeptide is poly-L-aspartic acid [poly(Asp)] having a molecular weight selected from a range of 15 kDa to 50 kDa.

As used herein the term “about” refers to a variation of plus/minus 10 percent.

Preferred hormones are cardiac hormones, hypothalamic hormones, neurotransmitter hormones, pigmentation hormones and vascular hormones.

Preferred cardiac hormones are brain natriuretic peptides (BNP) and human natriuretic peptides. A most preferred cardiac hormone is human brain natriuretic peptide.

Preferred hypothalamic hormones are leuteinizing hormone-releasing hormone (LHRH) and somatostatin. A most preferred LHRH is human LHRH.

Preferred neurotransmitter hormones are substance P and vasointestinal peptide (VIP).

A preferred pigmentation hormone is beta-melanocyte stimulating hormone (beta-MSH).

Preferred vascular hormones are vasopressin and vascular endothelial growth factor (VEGF). A preferred vascular endothelial growth factor is human vascular endothelial growth factor.

Preferred metabolic enzymes are matrix metalloproteinases (MMPs), acid phosphatases, aldolases, collagenases, holo-transferrins, galactosyltransferases (GSTase's), glutathione S-transferases (GSTs), and peroxidases.

Preferred matrix metalloproteinases are matrix metalloproteinase-9 (MMP9) and metalloproteinases of the same species as the subject.

A most preferred matrix metalloproteinase is matrix metalloproteinase-9 (MMP9) of the same species as the subject.

Preferred acid phosphatases are human acid phosphatases and semen-derived acid phosphatases. A most preferred acid phosphatase is human serum-derived acid phosphatase.

A preferred aldolase is muscle aldolase. A suitable muscle aldolase is rabbit muscle aldolase.

A preferred collagenase is Clostridium hystolyticum collagenase.

A preferred holo-transferrin is human holo-transferrin.

A preferred galactosyltransferase is bovine milk galactosyltransferase.

A preferred glutathione S-transferase is human glutathione S-transferase.

A preferred peroxidase is horseradish peroxidase (HRP).

Preferred microbial antigens are lipopolysaccharides, hemagglutinins, Streptococcal antigens and influenza antigens.

Preferred polysaccharides are polysaccharide type 4 (PS4) and streptococcal polysaccharides.

Preferred streptococcal polysaccharides are Streptococcus pneumoniae polysaccharides.

Most preferred microbial antigens are Streptococcus pneumoniae polysaccharide type 4 and influenza virus hemagglutinin.

Preferred molluscan antigens are limpet antigens and hemocyanin. A preferred limpet antigen is a keyhole limpet antigen. A most preferred molluscan antigen is a limpet hemocyanin (KLH).

Preferred nucleic acid antigens are double-stranded deoxyribonucleic acids (dsDNAs) and single-stranded deoxyribonucleic acids (ssDNAs). Suitable double-stranded DNA is calf-thymus derived double-stranded DNA. Preferred single-stranded DNAs are A-polyT-A and homopolymeric single-stranded DNAs. A preferred A-polyT-A is AT₁₈A and homopolymeric single-stranded DNAs. A preferred single-stranded DNA is poly-cytosine [poly(C)]. A preferred poly(C) is poly(C)₂₀.

Preferred plant antigens are wheat-derived antigens and gliadins. A most preferred plant antigen is wheat-derived gliadin.

Preferred plasma molecules are albumins, coagulation regulators, and lipoproteins.

Preferred albumins are serum albumin and ovalbumin. Preferred serum albumins are methylated albumins and mammalian albumins. A suitable albumin is methylated bovine serum albumin (methylated BSA).

Preferred coagulation regulators are plasmins and human coagulation regulators. A most preferred coagulation regulator is human plasmin.

Preferred lipoproteins are high density lipoprotein (HDL) and low density lipoprotein (LDL). Most preferred high density lipoprotein is human high density lipoprotein, respectively. Most preferred low density lipoprotein is human low density lipoprotein.

Preferred tissue antigens are cartilage antigens, nerve system molecules, and pancreatic molecules.

A preferred cartilage antigen is a cartilage extract. Preferred cartilage extracts are mammalian cartilage extracts and guanidine-extracted cartilage extract. A suitable cartilage extract is a guanidine-extracted bovine cartilage extract.

Preferred nerve system molecules are glutamic acid decarboxylases (GADs), myelin-associated oligodendrocytic basic proteins (MOBPs), myelin oligodendrocyte glycoproteins (MOGs) and synucleins.

Where the tissue antigen is GAD, a preferred at least a portion of a tissue antigen is a polypeptide which comprises at least one amino acid sequence selected from those set forth by SEQ ID NOs: 45 and 46. Preferred polypeptides which comprise at least one amino acid sequence selected from those set forth by SEQ ID NOs: 45 and 46 are the two polypeptides having the amino acid sequences set forth by SEQ ID NOs: 45 and 46, respectively.

Where the tissue antigen is MOBP, a preferred at least a portion of a tissue antigen is a polypeptide which comprises the amino acid sequence set forth by SEQ ID NO: 47. A preferred polypeptide which comprises the amino acid sequence set forth by SEQ ID NO: 47 is the polypeptide having the amino acid sequence set forth by SEQ ID NO: 47.

A preferred myelin oligodendrocyte glycoprotein is of the same species as the subject.

A preferred synuclein is of the same species as the subject.

Preferred pancreatic molecules are C-peptides, diabetes-associated peptide (DAP) amides, insulin chain B's, and glucagon.

A preferred C-peptide is a human C-peptide.

A preferred diabetes-associated peptide (DAP) amide is a human diabetes-associated peptide (DAP) amide.

A suitable insulin chain B is a bovine insulin chain B.

Descriptions of preferred/suitable antigen probes of the present invention, and sources thereof, are provided in Table 1 of the Examples section which follows.

Depending on the application and purpose, the method of the present invention may be practiced by determining the capacity of any of various isotypes of immunoglobulins of the subject to specifically bind any of various combinations of antigen probes of the present invention.

Preferably, the plurality of antigen probes comprises a number of distinct antigen probes selected from a range of 2 to 91 antigen probes. Depending on the application and purpose, the plurality of antigen probes may comprise a number of distinct antigen probes selected from a range of 2 to 5, 6 to 10, 11 to 15, 16 to 20, 21 to 25, 26 to 30, 31 to 35, 36 to 40, 41 to 45, 46 to 50, 51 to 55, 56 to 60, 61 to 65, 66 to 70, 71 to 75, 76 to 80, 81 to 85, or 86 to 91 antigen probes. Depending on the application and purpose, the plurality of antigen probes may comprise 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, or 91 distinct antigen probes.

The method may conveniently be practiced with the minimum number of antigen probes enabling a desired minimal level of diagnostic confidence. Alternately, the method may conveniently be practiced with a maximum number of antigen probes so as to provide a maximal level of diagnostic confidence. The combination of antigen probes needed to achieve a desired level of diagnostic confidence will be apparent to one of ordinary skill in the art in light of the teachings of the present invention, particularly in light of the teachings described in the Examples section which follows.

Preferably the method of the present invention is effected by determining the capacity of immunoglobulins of the IgG isotype and/or immunoglobulins of the IgM isotype of the subject to specifically bind antigen probes of the present invention. Most preferably, the method of the present invention is effected by determining the capacity of immunoglobulins of both the IgG and IgM isotypes of the subject to specifically bind antigen probes of the present invention.

According to teachings of the present invention, diagnosis of an immune disease such as type 1 diabetes, or predisposition thereto, in a subject can be made by determining the capacity of immunoglobulins of the IgG isotype of the subject to specifically bind antigen probes of a probe set of the present invention which comprises a plurality of antigen probes selected from group consisting of at least a portion of a human heat shock protein, at least a portion of a cardiac hormone, at least a portion of a neurotransmitter, at least a portion of a pigmentation hormone, at least a portion of a vascular hormone, at least a portion of an albumin, at least a portion of a lipoprotein, at least a portion of a nerve system molecule, at least a portion of a pancreatic molecule, at least a portion of a bacterial heat shock protein, at least a portion of a metabolic enzyme, at least a portion of a lipopolysaccharide, at least a portion of a molluscan antigen, at least a portion of a wheat antigen, and at least a portion of a nerve system molecule.

According to a preferred embodiment of the method of the present invention, a healthy subject's predisposition of to future onset of an immune disease such as type 1 diabetes is diagnosed by determining a capacity of IgG antibodies of the subject to specifically bind each antigen probe of an antigen probe set of the present invention which comprises the following antigen probes: at least a portion of a human 70 kDa heat shock protein, at least a portion of a cardiac hormone, at least a portion of a neurotransmitter, at least a portion of a pigmentation hormone, at least a portion of a vascular hormone, at least a portion of an albumin, at least a portion of a lipoprotein, at least a portion of a nerve system molecule, and at least a portion of a pancreatic molecule. Most preferably, in this context the antigen probe set comprises the following antigen probes: a polypeptide having the amino acid sequence set forth by SEQ ID NO: 24, a polypeptide having the amino acid sequence set forth by SEQ ID NO: 31, a polypeptide having the amino acid sequence set forth by SEQ ID NO: 37, BNP, VIP, beta-MSH, vasopressin, VEGF, methylated BSA, HDL, LDL, a polypeptide having the amino acid sequence set forth by SEQ ID NO: 46, DAP and glucagon. As is described in the Examples section below [refer, for example to Table 3 (“SA” subjects x predictive antigens)], a large majority of healthy subjects whose IgG antibodies are capable of specifically binding each antigen probe of the aforementioned antigen probe set are predisposed to future onset of an immune disease such as type 1 diabetes.

According to another preferred embodiment of the method of the present invention, a healthy subject's resistance to future onset of an immune disease such as type 1 diabetes is diagnosed by determining a capacity of IgG antibodies of the subject to specifically bind each antigen probe of an antigen probe set of the present invention which comprises the following antigen probes: a bacterial heat shock protein, at least a portion of a 60 kDa human heat shock protein, at least a portion of a 70 kDa human heat shock protein, at least a portion of a metabolic enzyme, at least a portion of a lipopolysaccharide, at least a portion of a molluscan antigen, at least a portion of a wheat antigen, and at least a portion of a nerve system molecule. Most preferably, in this context the antigen probe set comprises the following antigen probes: a polypeptide having an amino acid sequence set forth by SEQ ID NO: 1, a polypeptide having an amino acid sequence set forth by SEQ ID NO: 3, a polypeptide having an amino acid sequence set forth by SEQ ID NO: 14, a polypeptide having an amino acid sequence set forth by SEQ ID NO: 16, a polypeptide having an amino acid sequence set forth by SEQ ID NO: 20, a polypeptide having an amino acid sequence set forth by SEQ ID NO: 29, a polypeptide having an amino acid sequence set forth by SEQ ID NO: 35, HSP71, acid phosphatase, holo-transferrin, HRP, PS4, KLH, gliadin, and MOG. As is described in the Examples section below [refer, for example to Table 3 (“HA” subjects x predictive antigens)] a large majority of healthy subjects whose IgG antibodies are capable of specifically binding each antigen probe of the aforementioned antigen probe set will resist future onset of an immune disease such as type 1 diabetes.

According to a further preferred embodiment of the method of the present invention onset of an immune disease such as type 1 diabetes is diagnosed in a subject by determining a capacity of IgG antibodies of the subject to specifically bind each antigen probe of an antigen probe set of the present invention which comprises the following antigen probes: at least a portion of a 60 kDa human heat shock protein, at least a portion of a 70 kDa human heat shock protein, at least a portion of a cytokine receptor, at least a portion of a T-cell receptor, at least a portion of a metabolic enzyme, at least a portion of a coagulation regulator, at least a portion of a nerve system molecule, and at least a portion of a pancreatic molecule. Most preferably, in this context, the antigen probe set comprises the following antigen probes: a polypeptide having an amino acid sequence set forth by SEQ ID NO: 19, a polypeptide having an amino acid sequence set forth by SEQ ID NO: 22, a polypeptide having an amino acid sequence set forth by SEQ ID NO: 23, a polypeptide having an amino acid sequence set forth by SEQ ID NO: 25, a polypeptide having an amino acid sequence set forth by SEQ ID NO: 28, a polypeptide having an amino acid sequence set forth by SEQ ID NO: 33, a polypeptide having an amino acid sequence set forth by SEQ ID NO: 40, a polypeptide having an amino acid sequence set forth by SEQ ID NO: 41, a polypeptide having an amino acid sequence set forth by SEQ ID NO: 42, a polypeptide having an amino acid sequence set forth by SEQ ID NO: 44, aldolase, GSTase, plasmin, a polypeptide having an amino acid sequence set forth by SEQ ID NO: 45, a polypeptide having an amino acid sequence set forth by SEQ ID NO: 47, and C-peptide. As is described in the Examples section below [refer, for example to Table 3 (“SA” subjects x diagnostic antigens)], a large majority of subjects whose IgG antibodies are capable of specifically binding each antigen probe of the aforementioned antigen probe set display onset of an immune disease such as type 1 diabetes.

According to yet a further preferred embodiment of the method of the present invention, absence of an immune disease such as type 1 diabetes in a subject is diagnosed by determining a capacity of IgG antibodies of the subject to specifically bind each antigen probe of an antigen probe set of the present invention which comprises the following antigen probes: at least a portion of a bacterial heat shock protein, at least a portion of a human 60 kDa heat shock protein, at least a portion of a human 70 kDa heat shock protein, and at least a portion of a glutathione S-transferase. Most preferably, in this context the antigen probe set comprises the following antigen probes: a polypeptide having an amino acid sequence set forth by SEQ ID NO: 1, a polypeptide having an amino acid sequence set forth by SEQ ID NO: 4, a polypeptide having an amino acid sequence set forth by SEQ ID NO: 5, a polypeptide having an amino acid sequence set forth by SEQ ID NO: 7, a polypeptide having an amino acid sequence set forth by SEQ ID NO: 10, a polypeptide having an amino acid sequence set forth by SEQ ID NO: 12, a polypeptide having an amino acid sequence set forth by SEQ ID NO: 20, a polypeptide having an amino acid sequence set forth by SEQ ID NO: 34, and collagenase. As is described in the Examples section below [refer, for example to Table 3 (“HA” subjects x diagnostic antigens)] a large majority of subjects whose IgG antibodies are capable of specifically binding each antigen probe of the aforementioned antigen probe set do not display an immune disease such as type 1 diabetes.

According to further teachings of the present invention, diagnosis of an immune disease such as type 1 diabetes, or predisposition thereto, in a subject can be made by determining the capacity of immunoglobulins of the IgM isotype of the subject to specifically bind antigen probes of a probe set of the present invention which comprises a plurality of antigen probes selected from group consisting of at least a portion of a muscle structure protein, at least a portion of a human heat shock protein, at least a portion of a homopolymeric polypeptide, at least a portion of a hypothalamic hormone, at least a portion of a neurotransmitter, at least a portion of a matrix metalloproteinase, at least a portion of a viral antigen, at least a portion of a wheat antigen, at least a portion of an albumin, at least a portion of a lipoprotein, at least a portion of a crystallin molecule, at least a portion of a bacterial heat shock protein, at least a portion of a complement molecule, at least a portion of a cytokine, and at least a portion of a nucleic acid.

According to a preferred embodiment of the method of the present invention, a healthy subject's predisposition to future onset of an immune disease such as type 1 diabetes is diagnosed by determining a capacity of IgM antibodies of the subject to specifically bind each antigen probe of an antigen probe set of the present invention which comprises the following antigen probes: at least a portion of a muscle structure protein, at least a portion of a 70 kDa human heat shock protein, at least a portion of a homopolymeric polypeptide, at least a portion of a hypothalamic hormone, at least a portion of a neurotransmitter, at least a portion of a matrix metalloproteinase, at least a portion of a viral antigen, at least a portion of a wheat antigen, at least a portion of an albumin, and at least a portion of a lipoprotein. Most preferably, in this context the antigen probe set comprises the following antigen probes: troponin, a polypeptide having an amino acid sequence set forth by 21, a polypeptide having an amino acid sequence set forth by 24, a polypeptide having an amino acid sequence set forth by 26, a polypeptide having an amino acid sequence set forth by 32, a polypeptide having an amino acid sequence set forth by 36, a polypeptide having an amino acid sequence set forth by 37, a polypeptide having an amino acid sequence set forth by 39, poly(Asp), LHRH, somatostatin, substance P, MMP9, hemagglutinin, gliadin, and HDL. As is described in the Examples section below [refer, for example to Table 4 (SA subjects x predictive antigens)], a large majority of healthy subjects whose IgM antibodies are capable of specifically binding each antigen probe of the aforementioned antigen probe set are predisposed to future onset of an immune disease such as type 1 diabetes.

According to another preferred embodiment of the method of the present invention, a subject's resistance to future onset of an immune disease such as type 1 diabetes is diagnosed by determining a capacity of IgM antibodies of the subject to specifically bind each antigen probe of an antigen probe set of the present invention which comprises the following antigen probes: at least a portion of a crystallin, at least a portion of a bacterial heat shock protein, at least a portion of a human 60 kDa heat shock protein, at least a portion of a human 70 kDa heat shock protein, at least a portion of a complement molecule, at least a portion of a cytokine, and at least a portion of a nucleic acid. Most preferably, in this context the antigen probe set comprises the following antigen probes: alpha-crystallin, a polypeptide having an amino acid sequence set forth by SEQ ID NO: 2, a polypeptide having an amino acid sequence set forth by SEQ ID NO: 4, a polypeptide having an amino acid sequence set forth by SEQ ID NO: 6, a polypeptide having an amino acid sequence set forth by SEQ ID NO: 7, a polypeptide having an amino acid sequence set forth by SEQ ID NO: 8, a polypeptide having an amino acid sequence set forth by SEQ ID NO: 9, a polypeptide having an amino acid sequence set forth by SEQ ID NO: 15, a polypeptide having an amino acid sequence set forth by SEQ ID NO: 17, a polypeptide having an amino acid sequence set forth by SEQ ID NO: 35, C5, IL-4, IL-10, AT₁₈A, and poly(C). As is described in the Examples section below [refer, for example to Table 4 (“HA” subjects x predictive antigens)] a large majority of healthy subjects whose IgM antibodies are capable of specifically binding each antigen probe of the aforementioned antigen probe set will resist future onset of an immune disease such as type 1 diabetes.

According to further preferred embodiment of the method of the present invention, onset of an immune disease such as type 1 diabetes in a subject is diagnosed by determining a capacity of IgM antibodies of the subject to specifically bind each antigen probe of an antigen probe set of the present invention which comprises the following antigen probes: at least a portion of a human 70 kDa heat shock protein, at least a portion of a cytokine, at least a portion of a metabolic enzyme, at least a portion of a viral antigen, at least a portion of a molluscan antigen, at least a portion of an albumin, at least a portion of a lipoprotein, at least a portion of a cartilage antigen, and at least a portion of a nerve system molecule. Most preferably, in this context the antigen probe set comprises the following antigen probes: a polypeptide having an amino acid sequence set forth by SEQ ID NO: 26, a polypeptide having an amino acid sequence set forth by SEQ ID NO: 27, a polypeptide having an amino acid sequence set forth by SEQ ID NO: 29, a polypeptide having an amino acid sequence set forth by SEQ ID NO: 30, a polypeptide having an amino acid sequence set forth by SEQ ID NO: 33, a polypeptide having an amino acid sequence set forth by SEQ ID NO: 38, TNF-alpha, GST, hemagglutinin, KLH, dsDNA, ssDNA, OVA, HDL, cartilage extract, and synuclein. As is described in the Examples section below [refer, for example to Table 4 (“SA” subjects x diagnostic antigens)] a large majority of subjects whose IgM antibodies are capable of specifically binding each antigen probe of the aforementioned antigen probe set display an immune disease such as type 1 diabetes.

According to yet a further preferred embodiment of the method of the present invention, absence of an immune disease such as type 1 diabetes in a subject is diagnosed by determining a capacity of IgM antibodies of the subject to specifically bind each antigen probe of an antigen probe set of the present invention which comprises the following antigen probes: at least a portion of a bacterial heat shock protein, at least a portion of a human 60 kDa heat shock protein, at least a portion of a human 70 kDa heat shock protein, at least a portion of T-cell receptor, at least a portion of a lipopolysaccharide and at least a portion of a pancreatic molecule. Most preferably, in this context the antigen probe set comprises the following antigen probes: a polypeptide having an amino acid sequence set forth by SEQ ID NO: 6, a polypeptide having an amino acid sequence set forth by SEQ ID NO: 7, a polypeptide having an amino acid sequence set forth by SEQ ID NO: 11, a polypeptide having an amino acid sequence set forth by SEQ ID NO: 13, a polypeptide having an amino acid sequence set forth by SEQ ID NO: 18, a polypeptide having an amino acid sequence set forth by SEQ ID NO: 21, TCR beta-chain C2, and insulin chain B. As is described in the Examples section below [refer, for example to Table 4 (“HA” subjects x diagnostic antigens)] a large majority of subjects whose IgM antibodies are capable of specifically binding each antigen probe of the aforementioned antigen probe set do not display an immune disease such as type 1 diabetes.

Thus, the present invention provides an antigen probe set having utility in diagnosing an immune disease such as type 1 diabetes, or a predisposition thereto, in a subject.

As mentioned hereinabove, the method of the present invention may be effected by determining in any of various ways the capacity of the immunoglobulins to specifically bind the antigen probes. Preferably, determining the capacity of the immunoglobulins to specifically bind the antigen probes is performed using an antigen probe array-based method. Preferably, the array is incubated with suitably diluted serum of the subject so as to allow specific binding between antibodies contained in the serum and the immobilized antigen probes, washing out unbound serum from the array, incubating the washed array with a detectable label-conjugated ligand of antibodies of the desired isotype, washing out unbound label from the array, and measuring levels of the label bound to each antigen probe. Most preferably, determination of the capacity of immunoglobulins of a subject of the present invention to specifically bind antigen probes of the present invention is practiced as described in the Examples section which follows. Alternately, it will be well within the purview of the ordinarily skilled artisan to use any of various other commonly practiced methods enabling detection of antigen-antibody binding so as to determine the capacity of the immunoglobulins to specifically bind the antigen probes. Ample guidance for practicing array-based methods of determining the capacity of antibodies of a subject to specifically bind to antigens such as the antigen probes of the present invention is provided in the Examples section which follows and in the literature of the art (refer, for example, to Robinson W H. et al., 2002. Nat Med. 8:295-301; Robinson W H. et al., 2003. Nat Biotechnol. 21:1033-9; Glokler J, Angenendt P., 2003. Protein and antibody microarray technology. J Chromatogr B Analyt Technol Biomed Life Sci. 797:229-40; Valafar F., 2002. Pattern recognition techniques in microarray data analysis: a survey. Ann N Y Acad. Sci. 980:41-64; Wilson D S, Nock S., 2003. Recent developments in protein microarray technology. Angew Chem Int Ed Engl. 42:494-500; Templin M F. et al., 2002. Protein microarray technology. Drug Discov Today. 7:815-22; Forster T. et al., 2003. Experiments using microarray technology: limitations and standard operating procedures. J Endocrinol. 178(2):195-204).

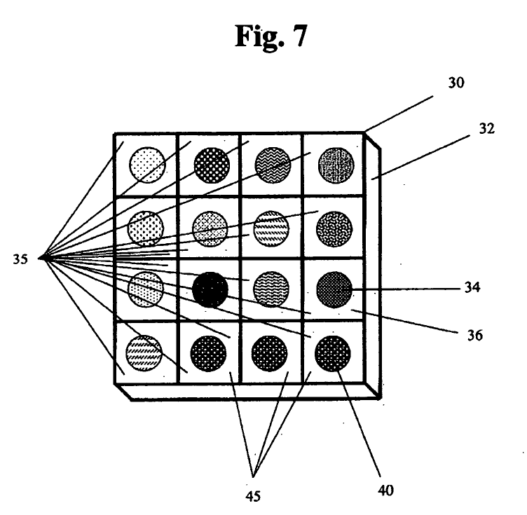

An example of an antigen probe array is illustrated in FIG. 7. Antigen probe array 30 includes a support 32 which can be fabricated from glass and shaped so as to form an upward-facing planar surface. Support 32 includes a plurality of addressable locations (each indicated by 35) which can be configured as wells, microwells, or areas delineated by grid etchings. Each antigen probe 34 of the antigen probe set of the present invention is immobilized to a specific addressable location 36 of antigen probe array 30. Such immobilization can be effected via covalent or non-covalent interactions between the antigen probes and the surface of the array support or between support bound linker molecules and the antigen probes. Preferably, a detectable label 40 is immobilized to each of a set of reference addressable locations (each indicated by 45) so as to provide a reference point for identifying each addressable location (indicated by 35).

Various types of antigen probe arrays may be used, depending on the application and purpose. Suitable types of antigen probe arrays for practicing the method of the present invention may be referred to in the art variously as protein/DNA microarrays, protein/DNA chips, or protein/DNA biochips. Large numbers of distinct antigen probes of the present invention, may be analyzed simultaneously using an antigen probe array of the present invention, allowing precise high throughput measurement of specific binding of immobilized antigen probes with a subject's antibodies.

Various methods have been developed for preparing arrays such as the antigen probe array of the present invention. State-of-the-art methods involves using a robotic apparatus to apply or “spot” distinct solutions containing antigen probes to closely spaced specific addressable locations on the surface of a planar support, typically a glass support, such as a microscope slide, which is subsequently processed by suitable thermal and/or chemical treatment to attach antigen probes to the surface of the support. Suitable supports may also include silicon, nitrocellulose, paper, cellulosic supports, and the like, and may be obtained from various commercial suppliers, for example, as indicated in the Examples section below.

Preferably, each antigen probe, or distinct subset of antigen probes of the present invention, which is attached to a specific addressable location of the array is attached independently to at least two, more preferably to at least three separate specific addressable locations of the array in order to enable generation of statistically robust data.

In addition to antigen probes of the antigen probe set, the array may advantageously include control antigen probes. Such control antigen probes may include normalization control probes. The signals obtained from the normalization control probes provide a control for variations in binding conditions, label intensity, “reading” efficiency and other factors that may cause the signal of a given binding antibody-probe ligand interaction to vary. For example, signals, such as fluorescence intensity, read from all other antigen probes of the antigen probe array are divided by the signal (e.g., fluorescence intensity) from the normalization control probes thereby normalizing the measurements. Normalization control probes can be bound to various addressable locations the antigen probe array to control for spatial variation in antibody-ligand probe efficiency. Preferably, normalization control probes are located at the corners or edges of the array to control for edge effects, as well as in the middle of the array.

One of ordinary skill in the art will possess the necessary expertise to obtain or produce an antigen probe of the present invention, such as a peptide antigen probe or a nucleic acid antigen probe. Ample guidance is provided in the literature of the art for obtaining molecules such as the antigen probes of the present invention (refer, for example, to the list of references in the introduction to the Examples section below, and to the extensive guidelines provided by The American Chemical Society (http://www.chemistry.org/portal/Chemistry). One of ordinary skill in the art, such as, for example, a chemist/biochemist, will possess the required expertise for practicing chemicaubiochemical techniques suitable for obtaining antigen probes of the present invention.

As used herein, the term “peptide” refers to a polypeptide which is composed of 50 amino acid residues or less. Peptides according to the present invention may include native peptides (either degradation products, synthetically synthesized peptides or recombinant peptides) and peptidomimetics (typically, synthetically synthesized peptides), such as peptoids and semipeptoids which are peptide analogs, which may have, for example, modifications rendering the peptides more stable for manipulation. Such modifications include, but are not limited to, N terminus modification, C terminus modification, peptide bond modification, including, but not limited to, CH2—NH, CH2—S, CH2—S═O, O═C—NH, CH2—O, CH2—CH2, S═C—NH, CH═CH or CF═CH, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992). For general guidance regarding chemical synthesis and modification of peptides, refer, for example to the extensive guidelines provided by The American Chemical Society (http://www.chemistry.org/portal/Chemistry).

Peptide bonds (—CO—NH—) within the peptide may be substituted, for example, by N-methylated bonds (—N(CH3)—CO—), ester bonds (—C(R)H—C—O—O—C(R)—N—), ketomethylen bonds (—C0-CH2—), α-aza bonds (—NH—N(R)—CO—), wherein R is any alkyl, e.g., methyl, carba bonds (—CH2—NH—), hydroxyethylene bonds (—CH(OH)—CH2—), thioamide bonds (—CS—NH—), olefinic double bonds (—CH═CH—), retro amide bonds (—NH—CO—), peptide derivatives (—N(R)—CH2—CO—), wherein R is the “normal” side chain, naturally presented on the carbon atom.

These modifications can occur at any of the bonds along the peptide chain and even at several (2-3) at the same time.

Natural aromatic amino acids, Trp, Tyr and Phe, may be substituted for synthetic non-natural acid such as TIC, naphthylelanine (Nol), ring-methylated derivatives of Phe, halogenated derivatives of Phe or o-methyl-Tyr.

In addition to the above, the peptides of the present invention may also include one or more modified amino acids or one or more non-amino acid monomers (e.g. fatty acids, complex carbohydrates etc).

As used herein in the specification and in the claims section below the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

Tables 5 below lists naturally occurring amino acids, and Table 6 below lists non-conventional or modified amino acids which can be present in the peptides of the present invention.

TABLE 5
Naturally occurring amino acids.

| | Three-Letter | One-letter |
|---|---|---|
| Amino Acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| Glycine | Gly | G |
| Histidine | His | H |
| Isoleucine | Iie | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid as above | Xaa | X |

TABLE 6
Non-conventional or modified amino acids.

| Non-conventional amino acid | Code | Non-conventional amino acid | Code |
|---|---|---|---|
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgin |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcyclopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cyclododeclglycine | Ncdod |
| D-α-methylalnine | Dnmala | N-cyclooctylglycine | Ncoct |
| D-α-methylarginine | Dnmarg | N-cyclopropylglycine | Ncpro |
| D-α-methylasparagine | Dnmasn | N-cycloundecylglycine | Ncund |
| D-α-methylasparatate | Dnmasp | N-(2,2-diphenylethyl)glycine | Nbhm |
| D-α-methylcysteine | Dnmcys | N-(3,3-diphenylpropyl)glycine | Nbhe |
| D-N-methylleucine | Dnmleu | N-(3-indolylyethyl) glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nva |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomo phenylalanine | Mhphe |
| L-α-methylisoleucine | Mile | N-(2-methylthioethyl)glycine | Nmet |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl)glycine | Nser |
| D-N-methylisoleucine | Dnmile | N-(imidazolylethyl)glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | mser | L-α-methylthreonine | Mthr |
| L-α-methylvaline | Mtrp | L-α-methyltyrosine | Mtyr |
| L-α-methylleucine | Mval Nnbhm | L-N-methylhomophenylalanine | Nmhphe |
| N-(N-(2,2-diphenylethyl) | | N-(N-(3,3-diphenylpropyl) | |
| carbamylmethyl-glycine | Nnbhm | carbamylmethyl(1)glycine | Nnbhe |
| 1-carboxy-1-(2,2-diphenylethylamino) | Nmbc | | |
| cyclopropane | | | |

The peptides of the present invention can be utilized in a linear or cyclic form.

A peptide can be either synthesized in a cyclic form, or configured so as to assume a cyclic structure under suitable conditions.

For example, a peptide according to the teachings of the present invention can include at least two cysteine residues flanking the core peptide sequence. In this case, cyclization can be generated via formation of S—S bonds between the two Cys residues. Side-chain to side chain cyclization can also be generated via formation of an interaction bond of the formula —(—CH2—)n—S—CH-2-C—, wherein n=1 or 2, which is possible, for example, through incorporation of Cys or homoCys and reaction of its free SH group with, e.g., bromoacetylated Lys, Om, Dab or Dap. Furthermore, cyclization can be obtained, for example, through amide bond formation, e.g., by incorporating Glu, Asp, Lys, Om, di-amino butyric (Dab) acid, di-aminopropionic (Dap) acid at various positions in the chain (—CO—NH or —NH—CO bonds). Backbone to backbone cyclization can also be obtained through incorporation of modified amino acids of the formulas H—N((CH2)n—COOH)—C(R)H—COOH or H—N((CH2)n—COOH)—C(R)H—NH2, wherein n=1-4, and further wherein R is any natural or non-natural side chain of an amino acid.

A nucleic acid antigen probe of the present invention may include any combination of any of various different types of nucleotide bases. Suitable nucleotide bases for preparing a nucleic acid antigen of the present invention may be selected from naturally occurring nucleotide bases such as adenine, cytosine, guanine, uracil, and thymine; and non-naturally occurring or non natural/synthetic nucleotide bases such as 8-oxo-guanine, 6-mercaptoguanine, 4-acetylcytidine, 5-(carboxyhydroxyethyl)uridine, 2′-O-methylcytidine, 5-carboxy-methylamino-methyl-2-thioridine, 5-carboxymethylaminomethyluridine, dihydro-uridine, 2′-O-methylpseudouridine, β,D-galactosylqueosine, 2′-O-methylguanosine, inosine, N6-isopentenyladenosine, 1-methyladenosine, 1-methylpseudouridine, 1-methylguanosine, 1-methylinosine, 2,2-dimethylguanosine, 2-methyladenosine, 2-methylguanosine, 3-methylcytidine, 5-methylcytidine, N6-methyladenosine, 7-methylguanosine, 5-methylaminomethyluridine, 5-methoxyaminomethyl-2-thiouridine, β,D-mannosylqueosine, 5-methoxycarbonylmethyluridine, 5-methoxy-uridine, 2-methylthio-N-6-isopentenyladenosine, N-((9-O-D-ribofuranosyl-2-methyl-thiopurine-6-yl)carbamoyl)threonine, N-((9-β-D-ribofuranosylpurine-6-yl)N-methyl-carbamoyl)threonine, unidine-5-oxyacetic acid methylester, uridine-5-oxyacetic acid, wybutoxosine, pseudouridine, queosine, 2-thiocytidine, 5-methyl-2-thiouridine, 2-thiouridine, 2-thiouridine, 5-methyluridine, N-((9-O-D-ribofuranosylpurine-6-yl)carbamoyl)threonine, 2′-O-methyl-5-methyluridine, 2′-O-methyluridine, wybutosine, and 3-(3-amino-3-carboxypropyl)uridine. Any nucleotide backbone may be employed, including DNA, RNA (although RNA is less preferred than DNA), modified sugars such as carbocycles, and sugars containing 2′ substitutions such as fluoro and methoxy. Any of the internucleotide bridging phosphate residues of a polynucleotide antigen probe of the present invention may be modified phosphates, such as methyl phosphonates, methyl phosphonothioates, phosphoromorpholidates, phosphoropiperazidates and phosphoramidates (for example, every other one of the internucleotide bridging phosphate residues may be modified as described).

The labeled subject-antibody ligands may be of any of various suitable types of antibody ligand. Preferably, the antibody ligand is an antibody which is capable of specifically binding the Fc portion of the antibodies of the subject used. For example, where the antibodies of the subject are of the IgG or of the IgM isotype, the antibody ligand is preferably an antibody capable of specifically binding to the Fc region of IgG or the Fc region of IgM antibodies of the subject, respectively.

The ligand of the antibodies of the subject may be conjugated to any of various types of detectable labels. Preferably the label is a fluorophore, most preferably Cy3. Alternately, the fluorophore may be any of various fluorophores, including Cy5, fluorescein isothiocyanate (FITC), phycoerythrin (PE), rhodamine, Texas red, and the like. As is described and illustrated in the Examples section below, the method may be performed using Cy3 as the fluorophore. Suitable fluorophore-conjugated antibodies specific for antibodies of a specific isotype are widely available from commercial suppliers and methods of their production are well established.

Thus, according to the present invention, there is provided an antigen probe array which is suitable for diagnosing an immune disease, or a predisposition thereto, in a subject.

Antibodies of the subject may be isolated for analysis of their antigen probe binding capacity in any of various ways, depending on the application and purpose. While the subject's antibodies may be suitably and conveniently in the form of serum or a dilution thereof, the antibodies may be subjected to any desired degree of purification prior to being tested for their capacity to specifically bind antigen probes.

The method of the present invention may be practiced using whole antibodies of the subject, or antibody fragments of the subject which comprise an antibody variable region.

Antibody fragments suitable for practicing the method of the present invention include a variable region of a light chain, a variable region of a heavy chain, a light chain, a heavy chain, an Fd fragment, and preferably antibody fragments comprising essentially whole variable regions of both light and heavy chains such as an Fv, a single chain Fv, an Fab, an Fab′, and an F(ab′)2.

Functional antibody fragments comprising whole or essentially whole variable regions of both light and heavy chains are defined as follows:
(i) Fv, defined as a genetically engineered fragment consisting of the variable region of the light chain and the variable region of the heavy chain expressed as two chains;
(ii) single chain Fv (“scFv”), a genetically engineered single chain molecule including the variable region of the light chain and the variable region of the heavy chain, linked by a suitable polypeptide linker.
(iii) Fab, a fragment of an antibody molecule containing a monovalent antigen-binding portion of an antibody molecule which can be obtained by treating whole antibody with the enzyme papain to yield the intact light chain and the Fd fragment of the heavy chain which consists of the variable and CH1 domains thereof;
(iv) Fab′, a fragment of an antibody molecule containing a monovalent antigen-binding portion of an antibody molecule which can be obtained by treating whole antibody with the enzyme pepsin, followed by reduction (two Fab′ fragments are obtained per antibody molecule); and
(v) F(ab′)2, a fragment of an antibody molecule containing a monovalent antigen-binding portion of an antibody molecule which can be obtained by treating whole antibody with the enzyme pepsin (i.e., a dimer of Fab′ fragments held together by two disulfide bonds).

The ordinarily skilled artisan will possess all necessary expertise to obtain from a subject of the present invention any type of antibody preparation/fraction at any desired level of purification according to any of various standard art methods. Ample guidance for obtaining and manipulating antibodies or antibody fragments suitable for practicing the method of the present invention is available in the literature of the art. [(see, for example, Harlow and Lane, “Antibodies: A Laboratory Manual”, Cold Spring Harbor Laboratory, New York, (1988)].

The method of the present invention can be used to diagnose in a subject any of various immune diseases, and predispositions thereto. Immune diseases according to the present invention include any disease which is associated with protective and/or pathogenic antibody responses. Specific types of immune diseases according to the present invention include autoimmune diseases, transplantation-related diseases, allergic diseases, infectious diseases, inflammation, and inflammation-associated diseases. Preferably, the immune disease is an autoimmune disease and/or a pancreatic disease, most preferably both of which. Most preferably the immune disease is type 1 diabetes.

Specific examples of immune diseases according to the present invention are listed hereinbelow.

Examples of autoimmune diseases associated with antibody mediated immune responses comprise rheumatoid diseases, rheumatoid autoimmune diseases, rheumatoid arthritis (Krenn V. et al., Histol Histopathol 2000 July; 15 (3):791), spondylitis, ankylosing spondylitis (Jan Voswinkel et al., Arthritis Res 2001; 3 (3): 189), systemic diseases, systemic autoimmune diseases, systemic lupus erythematosus (Erikson J. et al., Immunol Res 1998; 17 (1-2):49), sclerosis, systemic sclerosis (Renaudineau Y. et al., Clin Diagn Lab Immunol. 1999 March; 6 (2):156); Chan O T. et al., Immunol Rev 1999 June; 169:107), glandular diseases, glandular autoimmune diseases, pancreatic autoimmune diseases, diabetes, Type I diabetes (Zimmet P. Diabetes Res Clin Pract 1996 October; 34 Suppl:S125), thyroid diseases, autoimmune thyroid diseases, Graves' disease (Orgiazzi J. Endocrinol Metab Clin North Am 2000 June; 29 (2):339), thyroiditis, spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), myxedema, idiopathic myxedema (Mitsuma T. Nippon Rinsho. 1999 August; 57 (8):1759); autoimmune reproductive diseases, ovarian diseases, ovarian autoimmunity (Garza K M. et al., J Reprod Immunol 1998 February; 37 (2):87), autoimmune anti-sperm infertility (Diekman A B. et al., Am J Reprod Immunol. 2000 March; 43 (3):134), repeated fetal loss (Tincani A. et al., Lupus 1998; 7 Suppl 2:S107-9), neurodegenerative diseases, neurological diseases, neurological autoimmune diseases, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83), motor neuropathies (Komberg A J. J Clin Neurosci. 2000 May; 7 (3): 191), Guillain-Barre syndrome, neuropathies and autoimmune neuropathies (Kusunoki S. Am J Med Sci. 2000 April; 319 (4):234), myasthenic diseases, Lambert-Eaton myasthenic syndrome (Takamori M. Am J Med Sci. 2000 April; 319 (4):204), paraneoplastic neurological diseases, cerebellar atrophy, paraneoplastic cerebellar atrophy, non-paraneoplastic stiff man syndrome, cerebellar atrophies, progressive cerebellar atrophies, encephalitis, Rasmussen's encephalitis, amyotrophic lateral sclerosis, Sydeham chorea, Gilles de la Tourette syndrome, polyendocrinopathies, autoimmune polyendocrinopathies (Antoine J C. and Honnorat J. Rev Neurol (Paris) 2000 January; 156 (1):23); neuropathies, dysimmune neuropathies (Nobile-Orazio E. et al., Electroencephalogr Clin Neurophysiol Suppl 1999; 50:419); neuromyotonia, acquired neuromyotonia, arthrogryposis multiplex congenita (Vincent A. et al., Ann N Y Acad. Sci. 1998 May 13; 841:482), cardiovascular diseases, cardiovascular autoimmune diseases, atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), granulomatosis, Wegener's granulomatosis, arteritis, Takayasu's arteritis and Kawasaki syndrome (Praprotnik S. et al., Wien Klin Wochenschr 2000 Aug. 25; 112 (15-16):660); anti-factor VIII autoimmune disease (Lacroix-Desmazes S. et al., Semin Thromb Hemost. 2000; 26 (2):157); vasculitises, necrotizing small vessel vasculitises, microscopic polyangiitis, Churg and Strauss syndrome, glomerulonephritis, pauci-immune focal necrotizing glomerulonephritis, crescentic glomerulonephritis (Noel L H. Ann Med Interne (Paris). 2000 May; 151 (3):178); antiphospholipid syndrome (Flamholz R. et al., J Clin Apheresis 1999; 14 (4):171); heart failure, agonist-like beta-adrenoceptor antibodies in heart failure (Wallukat G. et al., Am J Cardiol. 1999 Jun. 17; 83 (12A):75H), thrombocytopenic purpura (Moccia F. Ann Ital Med Int. 1999 Apr-Jun; 14 (2):114); hemolytic anemia, autoimmune hemolytic anemia (Efremov D G. et al., Leuk Lymphoma 1998 January; 28 (3-4):285), gastrointestinal diseases, autoimmune diseases of the gastrointestinal tract, intestinal diseases, chronic inflammatory intestinal disease (Garcia Herola A. et al., Gastroenterol Hepatol. 2000 January; 23 (1):16), celiac disease (Landau Y E. and Shoenfeld Y. Harefuah 2000 Jan. 16; 138 (2):122), autoimmune diseases of the musculature, myositis, autoimmune myositis, Sjogren's syndrome (Feist E. et al., Int Arch Allergy Immunol 2000 September; 123 (1):92); smooth muscle autoimmune disease (Zauli D. et al., Biomed Pharmacother 1999 June; 53 (5-6):234), hepatic diseases, hepatic autoimmune diseases, autoimmune hepatitis (Manns M P. J Hepatol 2000 August; 33 (2):326) and primary biliary cirrhosis (Strassburg C P. et al., Eur J Gastroenterol Hepatol. 1999 June; 11 (6):595).

Examples of organ/tissue specific autoimmune diseases comprise cardiovascular diseases, rheumatoid diseases, glandular diseases, gastrointestinal diseases, cutaneous diseases, hepatic diseases, neurological diseases, muscular diseases, nephric diseases, diseases related to reproduction, connective tissue diseases and systemic diseases.

Examples of autoimmune cardiovascular diseases comprise atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), Wegener's granulomatosis, Takayasu's arteritis, Kawasaki syndrome (Praprotnik S. et al., Wien Klin Wochenschr 2000 Aug. 25; 112 (15-16):660), anti-factor VIII autoimmune disease (Lacroix-Desmazes S. et al., Semin Thromb Hemost. 2000; 26 (2): 157), necrotizing small vessel vasculitis, microscopic polyangiitis, Churg and Strauss syndrome, pauci-immune focal necrotizing and crescentic glomerulonephritis (Noel L H. Ann Med Interne (Paris). 2000 May; 151 (3):178), antiphospholipid syndrome (Flamholz R. et al., J Clin Apheresis 1999; 14 (4):171), antibody-induced heart failure (Wallukat G. et al., Am J Cardiol. 1999 Jun. 17; 83 (12A):75H), thrombocytopenic purpura (Moccia F. Ann Ital Med Int. 1999 Apr-Jun; 14 (2):114; Semple J W. et al., Blood 1996 May 15; 87 (10):4245), autoimmune hemolytic anemia (Efremov D G. et al., Leuk Lymphoma 1998 January; 28 (3-4):285; Sallah S. et al., Ann Hematol 1997 March; 74 (3):139), cardiac autoimmunity in Chagas' disease (Cunha-Neto E. et al., J Clin Invest 1996 Oct. 15; 98 (8):1709) and anti-helper T lymphocyte autoimmunity (Caporossi A P. et al., Viral Immunol 1998; 11 (1):9).

Examples of autoimmune rheumatoid diseases comprise rheumatoid arthritis (Krenn V. et al., Histol Histopathol 2000 July; 15 (3):791; Tisch R, McDevitt HO. Proc Natl Acad Sci units S A 1994 Jan. 18; 91 (2):437) and ankylosing spondylitis (Jan Voswinkel et al., Arthritis Res 2001; 3 (3): 189).

Examples of autoimmune glandular diseases comprise pancreatic disease, Type I diabetes, thyroid disease, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome diseases comprise autoimmune diseases of the pancreas, Type 1 diabetes (Castano L. and Eisenbarth G S. Ann. Rev. Immunol. 8:647; Zimmet P. Diabetes Res Clin Pract 1996 October; 34 Suppl:S125), autoimmune thyroid diseases, Graves' disease (Orgiazzi J. Endocrinol Metab Clin North Am 2000 June; 29 (2):339; Sakata S. et al., Mol Cell Endocrinol 1993 March; 92 (1):77), spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), idiopathic myxedema (Mitsuma T. Nippon Rinsho. 1999 August; 57 (8):1759), ovarian autoimmunity (Garza K M. et al., J Reprod Immunol 1998 February; 37 (2):87), autoimmune anti-sperm infertility (Diekman A B. et al., Am J Reprod Immunol. 2000 Mar; 43 (3):134), autoimmune prostatitis (Alexander R B. et al., Urology 1997 December; 50 (6):893) and Type I autoimmune polyglandular syndrome (Hara T. et al., Blood. 1991 Mar. 1; 77 (5):1127).

Examples of autoimmune gastrointestinal diseases comprise chronic inflammatory intestinal diseases (Garcia Herola A. et al., Gastroenterol Hepatol. 2000 January; 23 (1):16), celiac disease (Landau Y E. and Shoenfeld Y. Harefuah 2000 Jan. 16; 138 (2):122), colitis, ileitis and Crohn's disease.

Examples of autoimmune cutaneous diseases comprise autoimmune bullous skin diseases, such as, but are not limited to, pemphigus vulgaris, bullous pemphigoid and pemphigus foliaceus.

Examples of autoimmune hepatic diseases comprise hepatitis, autoimmune chronic active hepatitis (Franco A. et al., Clin Immunol Immunopathol 1990 March; 54 (3):382), primary biliary cirrhosis (Jones D E. Clin Sci (Colch) 1996 November; 91 (5):551; Strassburg C P. et al., Eur J Gastroenterol Hepatol. 1999 June; 11 (6):595) and autoimmune hepatitis (Manns M P. J Hepatol 2000 August; 33 (2):326).

Examples of autoimmune neurological diseases comprise multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83; Oshima M. et al., Eur J Immunol 1990 December; 20 (12):2563), neuropathies, motor neuropathies (Komberg A J. J Clin Neurosci. 2000 May; 7 (3):191); Guillain-Barre syndrome and autoimmune neuropathies (Kusunoki S. Am J Med Sci. 2000 April; 319 (4):234), myasthenia, Lambert-Eaton myasthenic syndrome (Takamori M. Am J Med Sci. 2000 April; 319 (4):204); paraneoplastic neurological diseases, cerebellar atrophy, paraneoplastic cerebellar atrophy and stiff-man syndrome (Hiemstra H S. et al., Proc Natl Acad Sci units S A 2001 Mar. 27; 98 (7):3988); non-paraneoplastic stiff man syndrome, progressive cerebellar atrophies, encephalitis, Rasmussen's encephalitis, amyotrophic lateral sclerosis, Sydeham chorea, Gilles de la Tourette syndrome and autoimmune polyendocrinopathies (Antoine J C. and Honnorat J. Rev Neurol (Paris) 2000 January; 156 (1):23); dysimmune neuropathies (Nobile-Orazio E. et al., Electroencephalogr Clin Neurophysiol Suppl 1999; 50:419); acquired neuromyotonia, arthrogryposis multiplex congenita (Vincent A. et al., Ann N Y Acad. Sci. 1998 May 13; 841:482), neuritis, optic neuritis (Soderstrom M. et al., J Neurol Neurosurg Psychiatry 1994 May; 57 (5):544) and neurodegenerative diseases.

Examples of autoimmune muscular diseases comprise myositis, autoimmune myositis and primary Sjogren's syndrome (Feist E. et al., Int Arch Allergy Immunol 2000 September; 123 (1):92) and smooth muscle autoimmune disease (Zauli D. et al., Biomed Pharmacother 1999 June; 53 (5-6):234).

Examples of autoimmune nephric diseases comprise nephritis and autoimmune interstitial nephritis (Kelly C J. J Am Soc Nephrol 1990 August; 1 (2):140).

Examples of autoimmune diseases related to reproduction comprise repeated fetal loss (Tincani A. et al., Lupus 1998; 7 Suppl 2:S107-9).

Examples of autoimmune connective tissue diseases comprise ear diseases, autoimmune ear diseases (Yoo T J. et al., Cell Immunol 1994 August; 157 (1):249) and autoimmune diseases of the inner ear (Gloddek B. et al., Ann N Y Acad Sci 1997 Dec. 29; 830:266).

Examples of autoimmune systemic diseases comprise systemic lupus erythematosus (Erikson J. et al., Immunol Res 1998; 17 (1-2):49) and systemic sclerosis (Renaudineau Y. et al., Clin Diagn Lab Immunol. 1999 March; 6 (2):156); Chan O T. et al., Immunol Rev 1999 June; 169:107).

Examples of transplantation-related diseases, but are not limited to, graft rejection, chronic graft rejection, subacute graft rejection, hyperacute graft rejection, acute graft rejection and graft-versus-host disease (GVHD).

Examples of allergic diseases comprise asthma, hives, urticaria, pollen allergy, dust mite allergy, venom allergy, cosmetics allergy, latex allergy, chemical allergy, drug allergy, insect bite allergy, animal dander allergy, stinging plant allergy, poison ivy allergy and food allergy.

Examples of antigen specific inflammatory diseases comprise inflammation associated with injuries, neurodegenerative diseases, ulcers, prosthetic implants, menstruation, septic shock, anaphylactic shock, toxic shock syndrome, cachexia, necrosis and gangrene; musculo-skeletal inflammations, and idiopathic inflammations.

Thus, the present invention provides an improved method of diagnosing in a subject an immune disease such as type 1 diabetes, or a predisposition thereto, relative to the prior art. As such the present invention enables improved prophylactic and therepeutic treatment of such a disease relative to the prior art.

It is expected that during the life of this patent many relevant medical diagnostic techniques will be developed and the scope of the term “diagnosing” is intended to include all such new technologies a priori.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W.H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

### Example 1

### Identification of Global Patterns of Antigenic Specificities of Antibodies Enabling Diagnosis of Diabetes, or Predisposition Thereto

Introduction: Satisfactory methods are presently unavailable for diagnosing the presence of, or predisposition to, numerous highly debilitating and/or lethal immune diseases, such as autoimmune, transplantation-related and allergic diseases. The ability to diagnose the predisposition to such diseases would provide valuable medical information which in many instances could be exploited, via appropriate behavioral, pharmacological and/or diagnostic measures, so as to prevent or delay disease onset, attenuate disease pathology, and/or optimize therapy. For example, in the case of type 1 diabetes, an autoimmune disease, appropriate behavioral or therapeutic regimens can be employed to prevent or delay disease onset and/or attenuate disease pathology. As well, close monitoring of signs and symptoms of type 1 diabetes could be used to initiate optimally early pharmacological control of glycemia. Similarly, the ability to correctly diagnose/stage type 1 diabetes would enable optimal medical management of the disease. One potentially potent strategy to diagnose occurrence or predisposition to such a disease would be to identify global patterns of antigenic specificities of antibodies correlating with such occurrence or predisposition, respectively. However, the prior art has generally failed to provide any such satisfactory correlations, in particular for type 1 diabetes. While reducing the present invention to practice, as described below, unexpected global patterns of antigenic specificities of antibodies characteristic of type 1 diabetes, and/or predisposition thereto, were empirically uncovered thereby overcoming the limitations of the prior art.

Materials and Methods:

Experiments described in this Example are published in Quintana F J. et al., 2004. Proc Natl Acad Sci USA. 101 Suppl 2:14615-21.

Mice: Male NOD mice were raised and maintained under pathogen-free conditions in the Animal Breeding Center of The Weizmann Institute of Science. The experiments were carried out under the supervision and guidelines of the Animal Welfare Committee. The mice were 4-weeks old at the start of the experiments. Nineteen mice were studied individually.

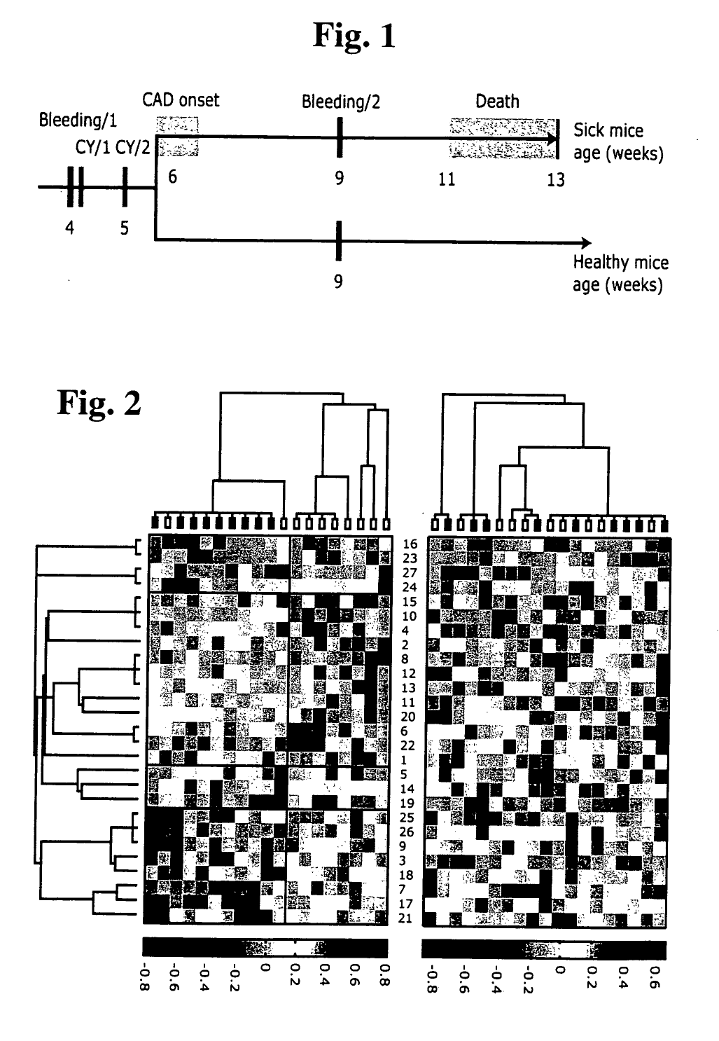

Cyclophosphamide-induced diabetes (CAD): Diabetes onset was accelerated and synchronized as previously described (Yasunami, R. & Bach, J. F., 1988. Eur J Immunol 18, 481-4) by two intraperitoneal injections of 200 mg/kg of cyclophosphamide (Sigma, Rehovot, Israel) given at the age of 4 weeks and again one week later. In the mouse colony from which the mice employed in the presently disclosed experiments were obtained, this treatment of NOD males leads to an incidence of diabetes about 50 percent (Ablamunits, V. et al., 1999. J Autoimmun 13, 383-92). The mice developing diabetes go on to die unless they are treated with insulin; those males that do not develop diabetes within I month after two injections of cyclophosphamide do not become diabetic later in life (data not shown). FIG. 1 is a schematic representation of the protocol. Four groups of mice were designated as follows: Mice that developed CAD are marked “sick” (S), and those that resisted CAD are marked “healthy” (H). The two groups are designated “sick before” (SB) and “healthy before” (HB) pre-CAD induction, and the two groups are designated “sick after” (SA) and “healthy after” (HA) post-CAD induction.

Diabetes monitoring: Blood glucose was measured weekly. A mouse was considered diabetic when its blood glucose concentration was higher than 13 millimolar on two consecutive examinations, tested using a Beckman Glucose Analyzer II (Beckman Instruments, Brea, Calif., USA). Of the 19 mice treated with cyclophosphamide, 9 developed diabetes and 10 remained healthy throughout a 2-month period of observation.

Serum harvesting: Serum samples were collected one day before the first injection of cyclophosphamide, and I month after the second injection. Blood was taken from the lateral tail vein, allowed to clot at room temperature and, after centrifugation, the sera were stored at minus 20 degrees centigrade.

Antigens: The antigens spotted on the microarray chips in these studies, which include proteins, synthetic peptides from the sequences of key proteins, nucleotides and phospholipids, are shown in Table 1.

TABLE 1
Antigen probes

| Antigen Probe Type | Antigen probe |
|---|---|
| Cell/tissue structure molecule | troponin (rabbit, muscle-derived; purchased from Sigma, Rehovot, |
| antigens - muscle structure | Israel, Cat. No. T3515) |
| protein | myosin (purchased from Sigma, Rehovot, Israel) |
| | tropomyosin (purchased from Sigma, Rehovot, Israel) |
| Cell/tissue structural antigens - | acid collagen (purchased from Sigma, Rehovot, Israel) |
| various | actin (purchased from Sigma, Rehovot, Israel) |
| | collagen I (purchased from Sigma, Rehovot, Israel) |
| | collagen IX (purchased from Sigma, Rehovot, Israel) |
| | collagen VI (purchased from Sigma, Rehovot, Israel) |
| | collagen X (purchased from Sigma, Rehovot, Israel) |
| | fibronectin (purchased from Sigma, Rehovot, Israel) |
| | gelsolin (purchased from Sigma, Rehovot, Israel) |
| | kinetesin (purchased from Sigma, Rehovot, Israel) |
| | laminin (purchased from Sigma, Rehovot, Israel) |
| | tubulin (purchased from Sigma, Rehovot, Israel) |
| | vimentin (purchased from Sigma, Rehovot, Israel) |
| | vitronectin (purchased from Sigma, Rehovot, Israel) |
| Heat shock protein antigens - | alpha-crystallin (purchased from Stressgen, Cat. No. Spp 235) |
| crystallin molecule | beta-crystallin (purchased from Sigma, Rehovot, Israel) |
| Heat shock protein antigens - | GroEL (E. coli, 65 kDa heat shock protein) fragment |
| bacterial, | [AAKDVKFGNDARVKMLRGVN (SEQ ID NO: 1)] peptide No. 01 |
| E. coli GroEL molecule* | GroEL (E. coli, 65 kDa heat shock protein) fragment |
| | [GPKGRNVVLDKSFGAPTITK (SEQ ID NO: 2)] peptide No. 03 |
| | GroEL (E. coli, 65 kDa heat shock protein) fragment |
| | [ATVLAQAIITEGLKAVAAGM (SEQ ID NO: 3)] peptide No. 07 |
| | GroEL (E. coli, 65 kDa heat shock protein) fragment |
| | [PCSDSKAIAQVGTISANSDE (SEQ ID NO: 4)] peptide No. 10 |
| | GroEL (E. coli, 65 kDa heat shock protein) fragment |
| | [ANSDETVGKLIAEAMDKVGK (SEQ ID NO: 5)] peptide No. 11 |
| | GroEL (E. coli, 65 kDa heat shock protein) fragment |
| | [RGYLSPYFINKPETGAVELE (SEQ ID NO: 6)] peptide No. 14 |
| | GroEL (E. coli, 65 kDa heat shock protein) fragment |
| | [AVELESPFILLADKKISNIR (SEQ ID NO: 7)] peptide No. 15 |
| | GroEL (E. coli, 65 kDa heat shock protein) fragment |
| | [ISNIREMLPVLEAVAKAGKP (SEQ ID NO: 8)] peptide No. 16 |
| | GroEL (E. coli, 65 kDa heat shock protein) fragment |
| | [KAGKPLLIIAEDVEGEALAT (SEQ ID NO: 9)] peptide No. 17 |
| | GroEL (E. coli, 65 kDa heat shock protein) fragment |
| | [EALATAVVNTIRGIVKVAAV (SEQ ID NO: 10)] peptide No. 18 |
| | GroEL (E. coli, 65 kDa heat shock protein) fragment |
| | [LGQAKRVVINKDTTTIIDGV (SEQ ID NO: 11)] peptide No. 22 |
| | GroEL (E. coli, 65 kDa heat shock protein) fragment |
| | [IIDGVGEEAAIQGRVAQIRQ (SEQ ID NO: 12)] peptide No. 23 |
| | GroEL (E. coli, 65 kDa heat shock protein; purchased from Sigma, |
| | Rehovot, Israel) |
| | GroEL (65 kDa heat shock protein, E. coli) peptide No. p2 |
| | GroEL (65 kDa heat shock protein, E. coli) peptide No. p4 |
| | GroEL (65 kDa heat shock protein, E. coli) peptide No. p5 |
| | GroEL (65 kDa heat shock protein, E. coli) peptide No. p6 |
| | GroEL (65 kDa heat shock protein, E. coli) peptide No. p8 |
| | GroEL (65 kDa heat shock protein, E. coli) peptide No. p9 |
| | GroEL (65 kDa heat shock protein, E. coli) peptide No. p12 |
| | GroEL (65 kDa heat shock protein, E. coli) peptide No. p13 |
| | GroEL (65 kDa heat shock protein, E. coli) peptide No. p19 |
| | GroEL (65 kDa heat shock protein, E. coli) peptide No. p20 |
| | GroEL (65 kDa heat shock protein, E. coli) peptide No. p21 |
| | GroEL (65 kDa heat shock protein, E. coli) peptide No. p24 |
| | GroEL (65 kDa heat shock protein, E. coli) peptide No. p25 |
| | GroEL (65 kDa heat shock protein, E. coli) peptide No. p26 |
| | GroEL (65 kDa heat shock protein, E. coli) peptide No. p28 |
| | GroEL (65 kDa heat shock protein, E. coli) peptide No. p29 |
| | GroEL (65 kDa heat shock protein, E. coli) peptide No. p30 |
| | GroEL (65 kDa heat shock protein, E. coli) peptide No. p31 |
| | GroEL (65 kDa heat shock protein, E. coli) peptide No. p32 |
| | GroEL (65 kDa heat shock protein, E. coli) peptide No. p33 |
| | GroEL (65 kDa heat shock protein, E. coli) peptide No. p34 |
| | GroEL (65 kDa heat shock protein, E. coli) peptide No. p35 |
| | GroEL (65 kDa heat shock protein, E. coli) peptide No. p36 |
| | GroEL (65 kDa heat shock protein, E. coli) peptide No. p37 |
| Heat shock protein antigens - | HSP60 (60 kDa heat shock protein, human) peptide 195-214 |
| human, HSP60 molecule* | [RKGVITVKDGKTLNDELEII (SEQ ID NO: 13)] peptide No. 14 |
| | HSP60 (60 kDa heat shock protein, human) peptide 225-244 |
| | [SPYFINTSKGQKCEFQDAYV (SEQ ID NO: 14)] peptide No. 16 |
| | HSP60 (60 kDa heat shock protein, human) peptide 271-290 |
| | [LVIIAEDVDGEALSTLVLNR (SEQ ID NO: 15)] peptide No. 19 |
| | HSP60 (60 kDa heat shock protein, human) peptide 316-335 |
| | [MAIATGGAVFGEEGLTLNLE (SEQ ID NO: 16)] peptide No. 22 |
| | HSP60 (60 kDa heat shock protein, human) peptide 376-395 |
| | [EQLDVTTSEYEKEKLNERLA (SEQ ID NO: 17)] peptide No. 26 |
| | HSP60 (60 kDa heat shock protein, human) peptide 466-485 |
| | [EIIKRTLKIPAMTIAKNAGV (SEQ ID NO: 18)] peptide No. 32 |
| | HSP60 (60 kDa heat shock protein, human) peptide 496-515 |
| | [QSSSEVGYDAMAGDFVNMVE (SEQ ID NO: 19)] peptide No. 34 |
| | HSP60 (60 kDa heat shock protein, human) peptide 437-460 |
| | [VLGGGVALLRVIPALDSLTPANED (SEQ ID NO: 20)] peptide |
| | No. p277 |
| | HSP60 (60 kDa heat shock protein, human, full-length, batch 1, |
| | recombinant, purified at I. R. Cohen's Lab) |
| | HSP60 (60 kDa heat shock protein, human, batch 2; purchased from |
| | Sigma, Rehovot, Israel) |
| | HSP60 (60 kDa heat shock protein, human) peptide No. p1 |
| | HSP60 (60 kDa heat shock protein, human) peptide No. p2 |
| | HSP60 (60 kDa heat shock protein, human) peptide No. p4 |
| | HSP60 (60 kDa heat shock protein, human) peptide No. p5 |
| | HSP60 (60 kDa heat shock protein, human) peptide No. p6 |
| | HSP60 (60 kDa heat shock protein, human) peptide No. p7 |
| | HSP60 (60 kDa heat shock protein, human) peptide No. p8 |
| | HSP60 (60 kDa heat shock protein, human) peptide No. p9 |
| | HSP60 (60 kDa heat shock protein, human) peptide No. p10 |
| | HSP60 (60 kDa heat shock protein, human) peptide No. p12 |
| | HSP60 (60 kDa heat shock protein, human) peptide No. p18 |
| | HSP60 (60 kDa heat shock protein, human) peptide No. p23 |
| | HSP60 (60 kDa heat shock protein, human) peptide No. p24 |
| | HSP60 (60 kDa heat shock protein, human) peptide No. p25 |
| | HSP60 (60 kDa heat shock protein, human) peptide No. p27 |
| | HSP60 (60 kDa heat shock protein, human) peptide No. p28 |
| | HSP60 (60 kDa heat shock protein, human) peptide No. p29 |
| | HSP60 (60 kDa heat shock protein, human) peptide No. p30 |
| | HSP60 (60 kDa heat shock protein, human) peptide No. p33 |
| | HSP60 (60 kDa heat shock protein, human) peptide No. p35 |
| | HSP60 (60 kDa heat shock protein, human) peptide No. p36 |
| | HSP60 (60 kDa heat shock protein, human) peptide No. p37 |
| | HSP60 (60 kDa heat shock protein, human) peptide No. p38 |
| Heat shock protein antigens - | HSP70 (70 kDa heat shock protein, human) peptide 16-35 |
| human, HSP70 molecule* | [SCVGVFQHGKVEIIANDQGN (SEQ ID NO: 21)] peptide No. 02 |
| | HSP70 (70 kDa heat shock protein, human) peptide 46-65 |
| | [DTERLIGDAAKNQVALNPQN (SEQ ID NO: 22)] peptide No. 04 |
| | HSP70 (70 kDa heat shock protein, human) peptide 76-95 |
| | [RKFGDPVVQSDMKHWPFQVI (SEQ ID NO: 23)] peptide No. 06 |
| | HSP70 (70 kDa heat shock protein, human) peptide 106-125 |
| | (SYKGETKAFYPEEISSMVLT (SEQ ID NO: 24)] peptide No. 08 |
| | HSP70 (70 kDa heat shock protein, human) peptide 121-140 |
| | [SMVLTKMKEIAEAYLGYPVT (SEQ ID NO: 25)] peptide No. 09 |
| | HSP70 (70 kDa heat shock protein, human) peptide 136-155 |
| | [GYPVTNAVITVPAYFNDSQR (SEQ ID NO: 26)] peptide No. 10 |
| | HSP70 (70 kDa heat shock protein, human) peptide 151-170 |
| | [NDSQRQATKDAGVIAGLNVL (SEQ ID NO: 27)] peptide No. 11 |
| | HSP70 (70 kDa heat shock protein, human) peptide 166-185 |
| | [GLNVLRIINEPTAAAIAYGL (SEQ ID NO: 28)] peptide No. 12 |
| | HSP70 (70 kDa heat shock protein, human) peptide 181-199 |
| | (IAYGLDRTGKGERNVLIFDL (SEQ ID NO: 29)] peptide No. 13 |
| | HSP70 (70 kDa heat shock protein, human) peptide 195-214 |
| | [LIFDLGGGTFDVSILTIDDG (SEQ ID NO: 30)] peptide No. 14 |
| | HSP70 (70 kDa heat shock protein, human) peptide 240-259 |
| | [FVEEFKRKHKKDISQNKRAV (SEQ ID NO: 31)] peptide No. 17 |
| | HSP70 (70 kDa heat shock protein, human) peptide 286-305 |
| | [SLFEGIDFYTSITRARFEEL (SEQ ID NO: 32)] peptide No. 20 |
| | HSP70 (70 kDa heat shock protein, human) peptide 316-335 |
| | [PVEKALRDAKLDKAQIHDLV (SEQ ID NO: 33)] peptide No. 22 |
| | HSP70 (70 kDa heat shock protein, human) peptide 331-350 |
| | [IHDLVLVGGSTRIPKVQKLL (SEQ ID NO: 34)] peptide No. 23 |
| | HSP70 (70 kDa heat shock protein, human) peptide 346-365 |
| | [VQKLLQDFFNGRDLNKSINP (SEQ ID NO: 35)] peptide No. 24 |
| | HSP70 (70 kDa heat shock protein, human) peptide 421-440 |
| | [PTKQTQIFTTYSDNQPGVLI (SEQ ID NO: 36)] peptide No. 29 |
| | HSP70 (70 kDa heat shock protein, human) peptide 436-455 |
| | [PGVLIQVYEGERAMTKDNNL (SEQ ID NO: 37)] peptide No. 30 |
| | HSP70 (70 kDa heat shock protein, human) peptide 466-485 |
| | [PAPGVPQIEVTFDIDANGIL (SEQ ID NO: 38)] peptide No. 32 |
| | HSP70 (70 kDa heat shock protein, human) peptide 481-500 |
| | [ANGILNVTATDKSTGKANKI (SEQ ID NO: 39)] peptide No. 33 |
| | HSP70 (70 kDa heat shock protein, human; purchased from Sigma, |
| | Rehovot, Israel) |
| | HSP70 (70 kDa heat shock protein, human) peptide No. p3 |
| | HSP70 (70 kDa heat shock protein, human) peptide No. p5 |
| | HSP70 (70 kDa heat shock protein, human) peptide No. p18 |
| | HSP70 (70 kDa heat shock protein, human) peptide No. p26 |
| | HSP70 (70 kDa heat shock protein, human) peptide No. p28 |
| | HSP70 (70 kDa heat shock protein, human) peptide No. p31 |
| | HSP70 (70 kDa heat shock protein, human) peptide No. p36 |
| | HSP70 (70 kDa heat shock protein, human) peptide No. p37 |
| Heat shock protein antigens - | HSP71 (Mycobacterium tuberculosis, 71 kDa heat shock protein, |
| bacterial, Mycobacterium | recombinant provided by Prof. R Van der Zee) |
| tuberculosis | |
| Heat shock protein antigens - | HSP65 (65 kDa heat shock protein; Provided by Prof. R. van der Zee) |
| various | HSP65 (65 kDa heat shock protein) peptide No. p3 |
| | HSP65 (65 kDa heat shock protein)peptide No. p180 |
| | HSP65 (65 kDa heat shock protein)peptide No. p278 |
| | 90 kDa heat shock protein alpha (HSP90 alpha, human; recombinant |
| | protein purified in laboratory of Prof. I. R. Cohen) |
| Immune system molecule | complement C5 (C5, human; purchased from Sigma, Rehovot, Israel, |
| antigens - complement molecule | Cat. No. C5788) |
| | complement C1Q (C1Q, human; purchased from Sigma, Rehovot, |
| | Israel) |
| | complement C9 (human; purchased from Sigma, Rehovot, Israel) |
| Immune system molecule | interleukin-4 (IL-4, mouse, recombinant; purchased from Pharmingen, |
| antigens - cytokine | San Diego, USA) |
| | interleukin-10 (IL-10, mouse, recombinant; purchased from |
| | Pharmingen, San Diego, USA) |
| | tumor necrosis factor-alpha (TNF-alpha; purchased from Pharmingen, |
| | San Diego, USA) |
| | interferon (IFN)-γ |
| | interleukin-12 (IL-12; purchased from Pharmingen, San Diego, USA) |
| | interleukin-2 (IL-2; purchased from Pharmingen, San Diego, USA) |
| | interleukin-5 (IL-5; purchased from Pharmingen, San Diego, USA) |
| | interleukin-6 (IL-6; purchased from Pharmingen, San Diego, USA) |
| | tumor necrosis factor receptor (TNFR; provided by Prof. D. Wallach) |
| Immune system molecule | interleukin-2 receptor (IL-2R) alpha-chain peptide |
| antigens - | [ASEESQGSRNSFPESEACPT (SEQ ID NO: 40)] peptide No. 2 |
| cytokine receptor | interleukin-2 receptor (IL-2R) beta-chain peptide |
| | [TTDTQKSTQSVYQENLAGHCR (SEQ ID NO: 41); synthesized at |
| | WIS] peptide No. 1 |
| | interleukin-2 receptor (IL-2R) alpha-chain fragment (synthesized at |
| | WIS) peptide No. 1 |
| Immune system molecule | rat T-cell receptor beta-chain C2 [TCR beta-chain C2, recombinant; |
| antigens - | recombinant protein purified in laboratory of I. R. Cohen] |
| T-cell receptor (TCR) | T-cell receptor (TCR) beta-chain peptide [DSPKPVTQNISAEAWGR |
| | (SEQ ID NO: 42); synthesized at WIS] peptide No. MED12 |
| | T-cell receptor (TCR) beta-chain fragment |
| | [VLVSALVLMAMVKKKNS (SEQ ID NO: 43); synthesized at |
| | WIS] peptide No. C2C |
| | T-cell receptor (TCR) beta-chain peptide [VTPPKVSLFEPSEAEIA |
| | (SEQ ID NO: 44); synthesized at WIS] peptide No. N12 |
| | T-cell receptor (TCR) beta-chain C1 (recombinant protein purified in |
| | laboratory of I. R. Cohen) |
| | T-cell receptor (TCR)-CDR3-pC9 (synthesized at WIS) |
| Homopolymeric polypeptide | poly-L-aspartic acid [poly(Asp); mol wt 15 to 50 kDa; synthesized at |
| antigens | WIS] |
| | poly-L-arginine [poly(Arg); synthesized at WIS) |
| | poly-L-glutamic acid [poly(Glu); synthesized at WIS] |
| | poly-L-lysine [poly(Lys); synthesized at WIS] |
| Hormone antigens - cardiac | brain natriuretic peptide (BNP, human; purchased from Sigma, |
| hormone | Rehovot, Israel, Cat. No. B5900) |
| Hormone antigens - | leuteinizing hormone-releasing hormone (LHRH, human; purchased |
| hypothalamic hormone | from Sigma, Rehovot, Israel, Cat. No. L7134) |
| | somatostatin (purchased from Sigma, Rehovot, Israel, Cat. No. S9129) |
| Hormone antigens - | substance P (purchased from Sigma, Rehovot, Israel, Cat. No. S6883) |
| neurotransmitter hormone | vasointestinal peptide (VIP; purchased from Sigma, Rehovot, Israel, |
| | Cat. No. V6130) |
| Hormone antigens - | beta-melanocyte stimulating hormone (beta-MSH, human; purchased |
| pigmentation hormone | from Sigma, Rehovot, Israel, Cat. No. M6513) |
| | alpha-melanocyte stimulating hormone (alpha-MSH; purchased from |
| | Sigma, Rehovot, Israel) |
| | gamma-melanocyte stimulating hormone (gamma-MSH; purchased |
| | from Sigma, Rehovot, Israel) |
| Hormone antigens - various | atrial natriuretic peptide (ANP; purchased from Sigma, Rehovot, |
| | Israel) |
| | big gastrin (purchased from Sigma, Rehovot, Israel) |
| | chorionic gonadotrophin (purchased from Sigma, Rehovot, Israel) |
| | corticotropin releasing factor (CRF; purchased from Sigma, Rehovot, |
| | Israel) |
| | endothelin 1 (purchased from Sigma, Rehovot, Israel) |
| | endothelin 2 (purchased from Sigma, Rehovot, Israel) |
| | growth hormone releasing factor (GNRH; purchased from Sigma, |
| | Rehovot, Israel) |
| | neuropeptide Y (purchased from Sigma, Rehovot, Israel) |
| | neurotensin (purchased from Sigma, Rehovot, Israel) |
| | oxytocin (purchased from Sigma, Rehovot, Israel) |
| | parathyroid hormone (PTH; purchased from Sigma, Rehovot, Israel) |
| | thyrocalcitonin (purchased from Sigma, Rehovot, Israel) |
| | thyroglobulin (purchased from Sigma, Rehovot, Israel) |
| Hormone antigens - vascular | vasopressin (AVP; purchased from Sigma, Rehovot, Israel, Cat. No. |
| hormone | V9879) |
| | vascular endothelial growth factor (VEGF, human, recombinant, 165 |
| | amino acid residue variant 293-VE; purchased from R&D Systems) |
| Metabolic enzyme antigens - | matrix metalloproteinase-9 (MMP9, mouse; purchased from Sigma, |
| matrix metalloproteinase | Rehovot, Israel, Cat. No. M1552) |
| (MMP) | matrix metalloproteinase-1 (MMP1; purchased from Sigma, Rehovot, |
| | Israel) |
| | matrix metalloproteinase-2 (MMP2; purchased from Sigma, Rehovot, |
| | Israel) |
| | matrix metalloproteinase-3 (MMP3; purchased from Sigma, Rehovot, |
| | Israel |
| Metabolic enzyme antigens - | acid phosphatase (human, prostatic, semen-derived; purchased from |
| various | Sigma, Rehovot, Israel, Cat. No. P1774) |
| | aldolase (rabbit, muscle; purchased from Sigma, Rehovot, Israel, Cat. |
| | No. A8811) |
| | collagenase (Clostridium hystolyticum; purchased from Sigma, |
| | Rehovot, Israel, Cat. No. C9891) |
| | holo-transferrin (human; purchased from Sigma, Rehovot, Israel, Cat. |
| | No. T4132) |
| | galactosyltransferase (GSTase; bovine, milk-derived; purchased from |
| | Sigma, Rehovot, Israel, Cat. No. G5507) |
| | glutathione S-transferase (GST, human; purchased from Sigma, |
| | Rehovot, Israel, Cat. No. G8642) |
| | horseradish peroxidase (HRP; purchased from Sigma, Rehovot, Israel, |
| | Cat. No. P6782) |
| | acetyl cholinesterase (purchased from Sigma, Rehovot, Israel) |
| | catalase (purchased from Sigma, Rehovot, Israel) |
| | enolase (purchased from Sigma, Rehovot, Israel) |
| | myeloperoxidase (purchased from Sigma, Rehovot, Israel) |
| | ribonuclease (purchased from Sigma, Rehovot, Israel) |
| | superoxide dismutase (SOD; purchased from Sigma, Rehovot, Israel) |
| | tyrosinase (purchased from Sigma, Rehovot, Israel) |
| | protease 133 (purchased from Sigma, Rehovot, Israel) |
| | caspase 3 (purchased from Sigma, Rehovot, Israel) |
| | caspase 8 (purchased from Sigma, Rehovot, Israel) |
| | annexin 33 kDa (purchased from Sigma, Rehovot, Israel) |
| | ubiquitin (purchased from Sigma, Rehovot, Israel) |
| | beta2-microglobulin (purchased from Sigma, Rehovot, Israel) |
| | alpha2-microglobulin (purchased from Sigma, Rehovot, Israel) |
| Microbial antigens - | polysaccharide type 4 [PS4, capsular, Streptococcus pneumoniae; |
| lipopolysaccharide | obtained from American Type Culture Collection (Manassas, VA)] |
| | lipopolysaccharide (LPS; E. coli; purchased from Sigma, Rehovot, |
| | Israel) |
| | lipopolysaccharide (LPS; P. gingivalis; provided by Prof. G. Nussbaum) |
| | lipopolysaccharide (LPS, S. minessota; purchased from Sigma, |
| | Rehovot, Israel) |
| Microbial antigens - viral | hemagglutinin (influenza virus; provided by Prof. R. Arnon) |
| antigen | |
| Microbial antigens - various | antigen D (S. typhimurium; purchased from Sigma, Rehovot, Israel) |
| | endoproteinase (Clu-c, S. aureus; provided by H. Amir-Kroll) |
| | glutathione-S-transferase (smGST, S. mansoni; purchased from |
| | Sigma, Rehovot, Israel) |
| | lipid A (S. minessotta; purchased from Sigma, Rehovot, Israel) |
| | pepstatin (purchased from Sigma, Rehovot, Israel) |
| | Pertussis toxin (PT; purchased from Sigma, Rehovot, Israel) |
| | purified protein derivative (PPD, M. tuberculosis; purchased from |
| | Sigma, Rehovot, Israel) |
| Molluscan antigens | keyhole limpet hemocyanin (KLH; purchased from Pierce, Oud |
| | Beijerland, The Netherlands, Cat. No. 77153) |
| Nucleic acid antigens | deoxyribonucleic acid (DNA) AT₁₈A (synthesized at WIS) |
| | deoxyribonucleic acid (DNA) poly(cytosine)₂₀ [poly(C); synthesized |
| | at WIS] |
| | double-stranded DNA (dsDNA, calf, thymus-derived; purchased from |
| | Sigma, Rehovot, Israel, Cat. No. D1501) |
| | single-stranded DNA (ssDNA, calf, thymus-derived; purchased from |
| | Sigma, Rehovot, Israel) |
| | deoxyribonucleic acid (DNA) CpG (synthesized at WIS) |
| | deoxyribonucleic acid (DNA) GpC (synthesized at WIS) |
| | deoxyribonucleic acid (DNA) TAAT (synthesized at WIS) |
| | deoxyribonucleic acid (DNA) poly(A) (synthesized at WIS) |
| | deoxyribonucleic acid (DNA) poly(G) (synthesized at WIS) |
| | deoxyribonucleic acid (DNA) poly(T) (synthesized at WIS] |
| Plant antigen - wheat antigen | gliadin (wheat; purchased from Sigma, Rehovot, Israel, Cat. No. |
| | G3375) |
| Plasma molecule antigens - | methylated bovine serum albumin (methylated BSA; purchased from |
| albumin | Sigma, Rehovot, Israel, Cat. No. A1009) |
| | ovalbumin (OVA, chicken, egg white-derived; purchased from Sigma, |
| | Rehovot, Israel, Cat. No. A5378) |
| | human serum albumin (purchased from Sigma, Rehovot, Israel) |
| | bovine serum albumin (BSA; purchased from Sigma, Rehovot, Israel) |
| | rat serum albumin (purchased from Sigma, Rehovot, Israel) |
| Plasma molecule antigens - | plasmin (human, plasma-derived, purchased from Sigma, Rehovot, |
| coagulation regulator | Israel) |
| | clotting factor II (factor II, human; purchased from Sigma, Rehovot, |
| | Israel) |
| | clotting factor X (factor X, human; purchased from Sigma, Rehovot, |
| | Israel) |
| | fibrin (purchased from Sigma, Rehovot, Israel) |
| | fibrinogen (purchased from Sigma, Rehovot, Israel) |
| | thrombin (purchased from Sigma, Rehovot, Israel) |
| Plasma molecule antigens - | high density lipoprotein (HDL; human, plasma-derived; purchased |
| lipoprotein | from Sigma, Rehovot, Israel, Cat. No. L5277) |
| | low density lipoprotein (LDL, human, plasma-derived; purchased |
| | from Sigma, Rehovot, Israel, Cat. No. L2139) |
| Plasma molecule antigens - | C-reactive protein (C-protein; purchased from Sigma, Rehovot, Israel) |
| various | defensin (purchased from Sigma, Rehovot, Israel) |
| | IgG (rat; purchased from Sigma, Rehovot, Israel) |
| | IgG (human; purchased from Sigma, Rehovot, Israel) |
| | IgM (human; purchased from Sigma, Rehovot, Israel) |
| Tissue antigens - cartilage | cartilage extract (bovine, articular cartilage-derived, guanidine- |
| | extract; purchased from Sigma, Rehovot, Israel, Cat. No. C5210) |
| Tissue antigens - nerve system | glutamic acid decarboxylase (GAD) peptide 509-528 |
| molecule | [IPPSLRTLEDNEERMSRLSK (SEQ ID NO: 45) peptide No. 34 |
| | (synthesized in WIS) |
| | glutamic acid decarboxylase (GAD) peptide 524-543 |
| | [SRLSKVAPVIKARMMEYGTT (SEQ ID NO: 46) peptide No. 35 |
| | (synthesized in WIS) |
| | myelin-associated oligodendrocytic basic protein (MOBP, human; |
| | provided by Prof. A. Ben Nun) peptide 78-89 [RPKQQPAAPPAVV |
| | (SEQ ID NO: 47)] |
| | myelin oligodendrocyte glycoprotein (MOG, mouse, recombinant; |
| | provided by Prof. A. Ben Nun) |
| | synuclein (mouse, recombinant; produced in laboratory of Prof. I. R. Cohen) |
| | brain extract (purchased from Sigma, Rehovot, Israel) |
| | beta amyloid (purchased from Sigma, Rehovot, Israel) |
| | contactin-associated protein 2 (CASPR2; recombinant protein purified |
| | in laboratory of E. Peles) |
| | glutamic acid decarboxylase (GAD; purchased from Sigma, Rehovot, |
| | Israel) |
| | myelin basic protein (MBP, guinea pig; purchased from Sigma, |
| | Rehovot, Israel) |
| | myelin basic protein (MBP, rat; provided by Prof. F. Mor) |
| | myelin oligodendrocyte glycoprotein (MOG, human; provided by |
| | Prof. A. Ben Nun) |
| | myelin oligodendrocyte glycoprotein (MOG, mouse; provided by |
| | Prof A. Ben Nun) peptide 35-55 |
| | myelin oligodendrocyte glycoprotein (MOG, mouse; provided by |
| | Prof A. Ben Nun) peptide 94-116 |
| | multi-PDZ domain protein 1 (MUPP; recombinant protein purified in |
| | laboratory of E. Peles) |
| | proteolipid protein (PLP; provided by Prof A. Ben Nun) |
| Tissue antigens - pancreatic | C-peptide (human; purchased from Sigma, Rehovot, Israel, |
| molecule | Cat. No. C5051) |
| | diabetes-associated peptide (DAP) amide (human; purchased from |
| | Sigma, Rehovot, Israel) |
| | insulin chain B (bovine; purchased from Sigma, Rehovot, Israel, Cat. |
| | No. 16383) |
| | glucagon (purchased from Sigma, Rehovot, Israel, Cat. No. G1774) |
| | insulin (purchased from Sigma, Rehovot, Israel) |
| | insulin chain A (purchased from Sigma, Rehovot, Israel) |
| | proinsulin (purchased from Sigma, Rehovot, Israel) |
| Tissue antigens - various | hemoglobin (purchased from Sigma, Rehovot, Israel) |
| | heparin (purchased from Sigma, Rehovot, Israel) |
| | histone IIA (purchased from Sigma, Rehovot, Israel) |
| | INAPC (recombinant protein purified in laboratory of E. Peles) |
| | insulin growth factor binding protein (IGFBP; recombinant protein |
| | purified in laboratory of E. Peles) |
| | protamine sulfate (purchased from Sigma, Rehovot, Israel) |
| | spectrin (purchased from Sigma, Rehovot, Israel) |

Antigen microarray preparation: Aliquots of solutions of antigens (1 mg/ml in PBS) were distributed in 384-well plates, and a robotic MicroGrid arrayer with solid spotting pins of 0.2 mm diameter (BioRobotics, Cambridge, UK) was used to spot the antigen solutions onto Arraylt SuperEpoxi Microarray Substrate slides (TeleChem International, Sunnyvale, Calif., USA). Each antigen was spotted in 2-8 replicates. The resultant antigen microarrays were stored at 4 degrees centigrade.

Antigen microarray analysis of antigenic specificities of antibodies: The antigen microarrays were washed with PBS and blocked for 1 hour at 37 degrees centigrade with 1 percent BSA, and incubated overnight at 4 degrees centigrade with a 1/5 dilution of the test serum in blocking buffer under a cover slip. The arrays were then washed and incubated at 37 degrees centigrade for 45 minutes with a 1/500 dilution of Cy3-conjugated goat anti-mouse IgG or IgM purchased from Jackson ImmunoResearch Labs. Inc. (West Grove, Pa., USA). The arrays were washed again, spun dried and scanned with a ScanArray 4000×scanner (GSI Luminomics, Billerica, Mass., USA). The results were recorded as TIFF files.

Image and data processing: The pixels that comprised each spot in the TIFF files and the local background were identified using histogram segmentation. The intensity of each spot and its local background were calculated as the mean of the corresponding pixel intensities. None of the spots containing antigens showed saturation. Technically faulty spots were identified by visual inspection and removed from the dataset. For each spot, the local background intensity was subtracted from the spot intensity. Spots with negative intensities were removed from the dataset. A log-base-2 transformation of the intensities resulted in reasonably constant variability at all intensity levels. The log-intensity of each antigen was calculated as the mean of the log-intensities of the replicates on each slide. The coefficient of variability (CV) between replicates on each array was around 30 percent.

To remove overall differences in intensities between arrays, the mean-log-intensity of each antigen on each array was scaled by subtracting the median of the mean-log-intensities of all antigens on the array. The scaled mean-log-intensity of an antigen is denoted the reactivity of the antigen.

The processed dataset consists of a matrix of IgG or IgM reactivities consisting of 266 rows and 38 columns (2 samples for each of 19 mice). Each column contains the reactivities measured on a given array and each row contains the reactivities measured for a given antigen over all arrays.

Additionally, the reactivity for each antigen measured prior to and following cyclophosphamide treatment in each mouse was combined into a log-ratio by subtracting the reactivity prior to treatment from the reactivity after treatment. This yielded a matrix of ratios with 266 rows and 19 columns.

Data analysis: The clustering of antigens and samples was based on the Superparamagnetic Clustering (SPC) algorithm (Blatt, M. et al., 1996. Physical Review Letters 76, 3251-3255) because it provides an inherent mechanism for identifying robust and stable clusters. The algorithm can be understood by an analogy to physics: as a parameter T (the temperature) is increased, the system undergoes phase transitions (for example, it melts). In the presently described case, T is increased from 0 (all objects form one cluster) to Tₘₐₓ (each object forms a separate cluster). The break up of larger clusters into smaller sub-clusters is governed by the structure of the data: similar objects tend to stay together over a large increase in T, while less similar objects break apart more easily. The range of T's for which a given cluster remains unchanged, denoted by ΔT, is used as a stability measure for the cluster. As the measure of similarity between objects, Euclidean distance for both samples and antigens was used. Since the antigen reactivities (or ratios) were first row-centered and normalized before being clustered, their squared distance is proportional to 1-r, where r is the correlation coefficient. The correlation coefficient captures similarity in shape and the Euclidean distance captures similarity in magnitude.

To determine subsets of the 266 antigen that would separate the sick and healthy mice, the Wilcoxon rank-sum test [Wilcox, R. R. (2001) Fundamentals of modern statistical methods: substantially improving power and accuracy (Springer-Verlag, New York] was used. This test is non-parametric; it is robust to outliers. One antigen is tested at a time, replacing the reactivities (or ratios) with ranks according to their magnitude: 1 for the smallest, 2 for the second smallest, and so on. The p-values found using this method were higher than 0.01; no single antigen was found to significantly discriminate between the two groups when the Bonferroni-correction [Stekel, D. (2003) Microarray Bioinformatics (Cambridge University Press, Cambridge)] or the False Discovery Rate method (Benjamini, Y. & Hochberg, Y., 1995. J. R. Stat. Soc. 57, 289-300) was applied. This means that the signal produced by any single antigen is unable to separate the sick from the healthy mice. Separability might be achieved, if at all, by using reactivity (or ratio) profiles defined over several antigens. To capture a collective effect of several antigens, the 27 antigens (10 percent of the 266 antigens in the study) with the lowest p-values were selected, and investigated as to how well they could separate sick from healthy mice, and which antigens showed correlated behavior over the samples, by applying two-way SPC. This gives an unsupervised clustering of the subset of antigens and of the samples. The clusters of samples found using this method were evaluated for their stability ΔT, specificity, and sensitivity. Specificity is the proportion of sick mice in the “sick cluster”; sensitivity is the proportion of the sick mice in the “sick cluster” compared to all the sick mice in the study.

Statistical significance: To obtain a measure of the significance of the separation between sick and healthy mice using the method outlined above, the following test was performed: From the group of healthy mice, 5 of the samples were picked at random, and similarly, for the group of sick mice 4 of the samples were randomly picked. These 9 samples were labeled as “type A”. The remaining samples were labeled as “type B”. It was hypothesized that there should be no clear separation between these randomized types: the Wilcoxon rank-sum test was used to identify the 27 antigens that differentiate best groups A and B. Next, the mice are clustered in the space of these 27 antigens, and stable, specific, and sensitive clusters are sought using SPC. The test was performed 1000 times on different randomized groups and recorded the stability, specificity, and sensitivity of the resulting clusters. The proportion of random clusters manifesting these features to the same or to a better degree than the actual cluster establishes the p-value of the actual cluster.

Experimental Results:

Selection of informative IgG-reactive antigens for pre-CAD mice: It was previously reported that coupled two-way clustering (CTWC) could be used to successfully separate human subjects already diabetic from healthy persons (Quintana, F. J. et al. 2003. J Autoimmun 21, 65-75). In the CAD mouse study done here, however, only a few of the clusters of co-regulated antigens using the CTWC technique separated the SB and HB mice, and then only for a subset of the mice. A different approach was therefore taken. Based on the sera taken before cyclophosphamide treatment, Table 2, antigen list I tabulates the 27 antigens that separate best between the sera of the 10 mice that resisted the induction of CAD (HB) and the 9 mice that developed CAD (SB) (using the Wilcoxon rank-sum test and taking the 10 percent with lowest p-values).

TABLE 2
Discriminating sets of IgG-reactive antigens.

| | | | Antigen | | |
|---|---|---|---|---|---|
| Antigen probe | | Antigen | List | | |
| type | Antigen probe | ID no.* | I | II | III |
| Heat shock protein | GroEL (E. coli, 65 kDa heat shock protein) fragment | 18 | H | H | |
| antigens - E. coli | [AAKDVKFGNDARVKMLRGVN (SEQ ID NO: 1)] | | | | |
| GroEL | GroEL (E. coli, 65 kDa heat shock protein) fragment | 9 | H | | |
| | [ATVLAQAIITEGLKAVAAGM (SEQ ID NO: 3)] | | | | |
| | GroEL (E. coli, 65 kDa heat shock protein) fragment | 35 | | H | |
| | [PCSDSKAIAQVGTISANSDE (SEQ ID NO: 4)] | | | | |
| | GroEL (E. coli, 65 kDa heat shock protein) fragment | 31 | | H | H |
| | [ANSDETVGKLIAEAMDKVGK (SEQ ID NO: 5)] | | | | |
| | GroEL (E. coli, 65 kDa heat shock protein) fragment | 47 | | H | |
| | [AVELESPFILLADKKISNIR (SEQ ID NO: 7)] | | | | |
| | GroEL (E. coli, 65 kDa heat shock protein) fragment | 51 | | H | |
| | [ISNIREMLPVLEAVAKAGKP (SEQ ID NO: 8)] | | | | |
| | GroEL (E. coli, 65 kDa heat shock protein) fragment | 44 | | H | |
| | [EALATAVVNTIRGIVKVAAV (SEQ ID NO: 10)] | | | | |
| | GroEL (E. coli, 65 kDa heat shock protein) fragment | 37 | | H | H |
| | [IIDGVGEEAAIQGRVAQIRQ (SEQ ID NO: 12)] | | | | |
| | GroEL (65 kDa heat shock protein, E. coli) peptide No. 25 | 53 | | | H |
| | GroEL (65 kDa heat shock protein, E. coli) peptide No. 28 | 56 | | | H |
| Heat shock protein | HSP60 (60 kDa heat shock protein, human) peptide 225-244 | 21 | H | | S |
| antigens - human | [SPYFINTSKGQKCEFQDAYV (SEQ ID NO: 14)] | | | | |
| HSP60 | H5P60 (60 kDa heat shock protein, human) peptide 316-335 | 17 | H | S | S |
| | [MAIATGGAVFGEEGLTLNLE (SEQ ID NO: 16)] | | | | |
| | HSP60 (60 kDa heat shock protein, human) peptide No. p30 | 55 | | | S |
| | HSP60 (60 kDa heat shock protein, human) peptide 496-515 | 36 | | S | S |
| | [QSSSEVGYDAMAGDFVNMVE (SEQ ID NO: 19)] | | | | |
| | HSP60 (60 kDa heat shock protein, human) peptide No. p35 | 59 | | | H |
| | HSP60 (60 kDa heat shock protein, human) peptide 437-460 | 26 | H | H | |
| | [VLGGGVALLRVIPALDSLTPANED (SEQ ID NO: 20)] | | | | |
| Heat shock protein | HSP70 (70 kDa heat shock protein, human) peptide 46-65 | 48 | | S | |
| antigens - human | [DTERLIGDAAKNQVALNPQN (SEQ ID NO: 22)] | | | | |
| HSP70 | HSP70 (70 kDa heat shock protein, human) peptide 76-95 | 46 | | S | S |
| | [RKFGDPVVQSDMKHWPFQVI (SEQ ID NO: 23)] | | | | |
| | HSP70 (70 kDa heat shock protein, human) peptide 106-125 | 10 | S | | H |
| | [SYKGETKAFYPEEISSMVLT (SEQ ID NO: 24)] | | | | |
| | HSP70 (70 kDa heat shock protein, human) peptide 121-140 | 32 | | S | |
| | [SMVLTKMKEIAEAYLGYPVT (SEQ ID NO: 25)] | | | | |
| | HSP70 (70 kDa heat shock protein, human) peptide 166-185 | 38 | | H | |
| | [GLNVLRIINEPTAAAIAYGL (SEQ ID NO: 28)] | | | | |
| | HSP70 (70 kDa heat shock protein, human) peptide 181-199 | 7 | H | S | S |
| | [IAYGLDRTGKGERNVLIFDL (SEQ ID NO: 29)] | | | | |
| | HSP70 (70 kDa heat shock protein, human) peptide 240-259 | 22 | S | | |
| | [FVEEFKRKHKKDISQNKRAV (SEQ ID NO: 31)] | | | | |
| | HSP70 (70 kDa heat shock protein, human) peptide 316-335 | 28 | | S | S |
| | [PVEKALRDAKLDKAQIHDLV (SEQ ID NO: 33)] | | | | |
| | HSP70 (70 kDa heat shock protein, human) peptide 331-350 | 49 | | H | |
| | [IHDLVLVGGSTRIPKVQKLL (SEQ ID NO: 34)] | | | | |
| | HSP70 (70 kDa heat shock protein, human) peptide 346-365 | 14 | H | | |
| | [VQKLLQDFFNGRDLNKSINP (SEQ ID NO: 35)] | | | | |
| | HSP70 (70 kDa heat shock protein, human) peptide 436-455 | 15 | S | | |
| | [PGVLIQVYEGERAMTKDNNL (SEQ ID NO: 37)] | | | | |
| Heat shock protein | HSP71 (Mycobacterium tuberculosis) | 19 | H | | |
| antigens - M. | | | | | |
| tuberculosis | | | | | |
| Immune system | interleukin-2 receptor (IL-2R) alpha-chain peptide | 34 | | S | |
| antigens - | [ASEESQGSRNSFPESEACPT (SEQ ID NO: 40)] | | | | |
| cytokine receptor | interleukin-2 receptor (IL-2R) beta-chain peptide | 45 | | S | |
| | [TTDTQKSTQSVUQENLAGHCR (SEQ ID NO: 41)] | | | | |
| Immune system | T-cell receptor (TCR) beta-chain peptide | 30 | | 5 | |
| antigens - | [DSPKPVTQNISAEAWGR (SEQ ID NO: 42)] | | | | |
| T-cell receptor | T-cell receptor (TCR) beta-chain peptide | 43 | | S | S |
| (TCR) | [VTPPKVSLFEPSEAEIA (SEQ ID NO: 44)] | | | | |
| Hormone antigens - | brain natriuretic peptide (BNP)-32 (human) | 20 | S | | |
| cardiac | | | | | |
| Hormone antigens - | somatostatin | 54 | | | H |
| hypothalamic | | | | | |
| Hormone antigens - | vasointestinal peptide (VIP) | 13 | S | | H |
| neurotransmitter | | | | | |
| Hormone antigens - | beta-melanocyte stimulating hormone (beta-MSH, human) | 2 | S | | H |
| pigmentation | | | | | |
| Hormone antigens - | vascular endothelial growth factor (VEGF, human, | 8 | S | | |
| vascular | recombinant, 165 amino acid residue variant 293-VE) | | | | |
| | vasopressin (AVP) | 6 | S | | H |
| Metabolic enzyme | acid phosphatase | 3 | H | | |
| antigens - various | aldolase | 33 | | S | |
| | collagenase | 39 | | H | |
| | holo-transferrin | 27 | H | | S |
| | GSTase | 50 | | H | |
| Microbial antigens - | PS4 | 16 | H | | |
| bacterial | | | | | |
| Molluscan | KLH | 25 | H | | S |
| antigens | | | | | |
| Plasma antigens - | human serum albumin | 58 | | | S |
| albumin | methylated BSA | 4 | S | | |
| Plasma antigens - | plasmin | 42 | | S | |
| coagulation | | | | | |
| regulator | | | | | |
| Plasma antigens- | HDL | 5 | S | | |
| lipoprotein | LDL | 1 | S | | H |
| Structural antigens - | vimentin | 57 | | | H |
| various | | | | | |
| Tissue antigens - | cartilage extract | 52 | | | S |
| cartilage | | | | | |
| Tissue antigens - | GAD peptide 509-528 [IPPSLRTLEDNEERMSRLSK (SEQ | 29 | | S | S |
| nerve system | ID NO: 45) | | | | |
| molecule | MOBP peptide 78-89 [RPKQQPAAPPAVV (SEQ ID NO: | 40 | | S | S |
| | 47)] | | | | |
| | MOG | 24 | H | | |
| Tissue antigens - | C-peptide | 41 | | S | |
| pancreatic | DAP | 11 | S | | |
| molecule | glucagon | 12 | S | | H |
| Tissue antigens - | gliadin | 23 | H | | |
| wheat | | | | | |

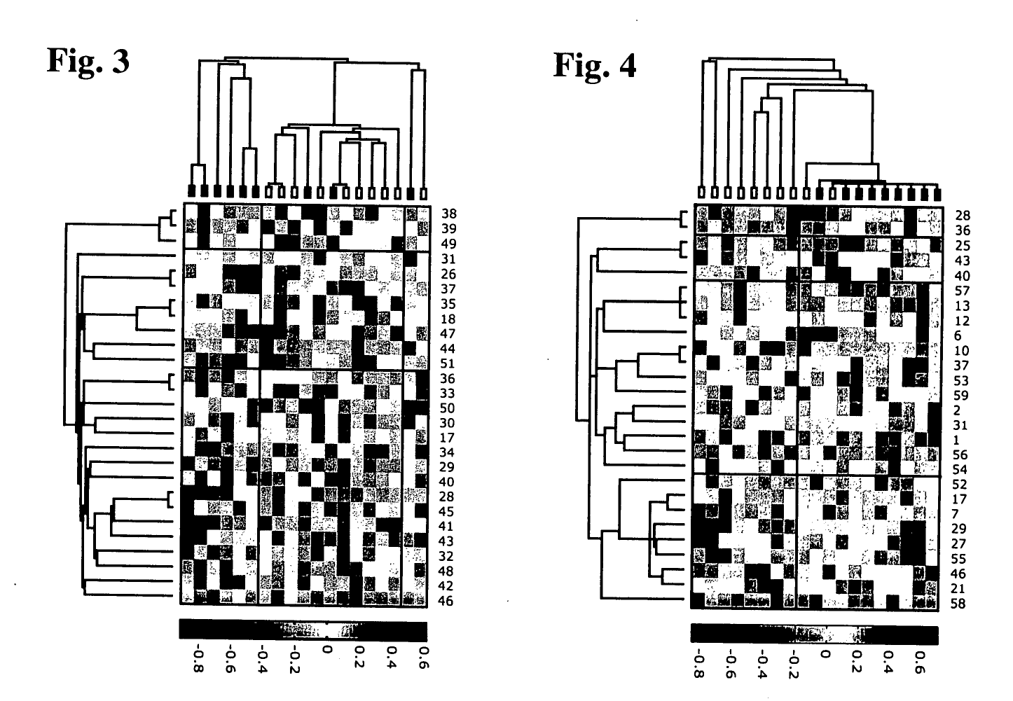

*Antigen ID no. corresponds to the antigen numbering scheme employed in FIGS. 2-5. In the antigen lists S and H respectively designate the sick and healthy group of mice in which the reactivity to the antigen (or pre-CAD/post-CAD ratio) was relatively the highest;
Antigen List I refers to the 27 antigens selected by rank-sum from the HB and SB groups pre-CAD.
Antigen List II refers to the 27 antigens selected by rank-sum for the HA and SA groups post-CAD.
Antigen List III refers to the 27 antigens selected by rank-sum from the pre-CAD/post-CAD ratios.

FIG. 2 (left panel) shows the two-way SPC of these antigens. The mice susceptible to future CAD induction are denoted by the filled rectangles at the top of the clustering box; the mice resistant to future CAD induction are denoted by the empty rectangles. The 27 antigens are clustered at the rows, and identified by number (see Table 2). It can be seen that all 9 mice that were found later to be susceptible to CAD (SB) could be separated from 8 of the 10 mice that were later found to resist CAD (HB); the SB mice manifested relatively elevated IgG reactivity to the top 19 antigens in FIG. 2 (left panel) while the HB mice manifested relatively elevated IgG reactivity to the remaining 8 antigens. The clustering separation was significant (p<0.017; only 17 of 1000 randomly generated groups showed results comparable to the actual data set). Thus mice susceptible to CAD could be distinguished by their patterns of antigenic specificities IgG serum antibodies in mice resistant to CAD, even before cyclophosphamide was administered to the mice.

Selection of informative IgG-reactive antigens for post-CAD mice: The 27 antigens effective in pre-CAD clustering were then used to analyze the patterns of antigenic specificities of IgG antibodies in the diabetic and healthy mice post-CAD. Surprisingly, these 27 antigens failed to discriminate between the two groups of mice; the obvious pre-CAD clusters seen in FIG. 2 (left panel), dispersed when the same antigens were used to cluster the post-CAD sera; compare the right and left panels in FIG. 2. For this reason, other sets of antigens which might be more informative post-CAD were tested. The 27 antigens listed in Table 2 List II was generated by performing the Wilcoxon rank-sum test on the reactivities measured post-CAD (HA and SA samples). A third set of 27 antigens was generated by performing the Wilcoxon rank-sum test on the ratios by which each antigen changed post-CAD/pre-CAD. The ratios provide information on reactivity changes toward the antigen. These antigens are shown in Table 2 List III.

FIGS. 3 and 4 show that the List II and the List III antigens could indeed separate between the healthy and diabetic mice post-CAD: specificity up to 82 percent and sensitivity up to 100 percent (p=0.065). Thus, the IgG repertoires of the pre-CAD and post-CAD groups of healthy and sick mice could be clustered, but the informative patterns of reactivity required modified sets of antigens to identify discriminating patterns.

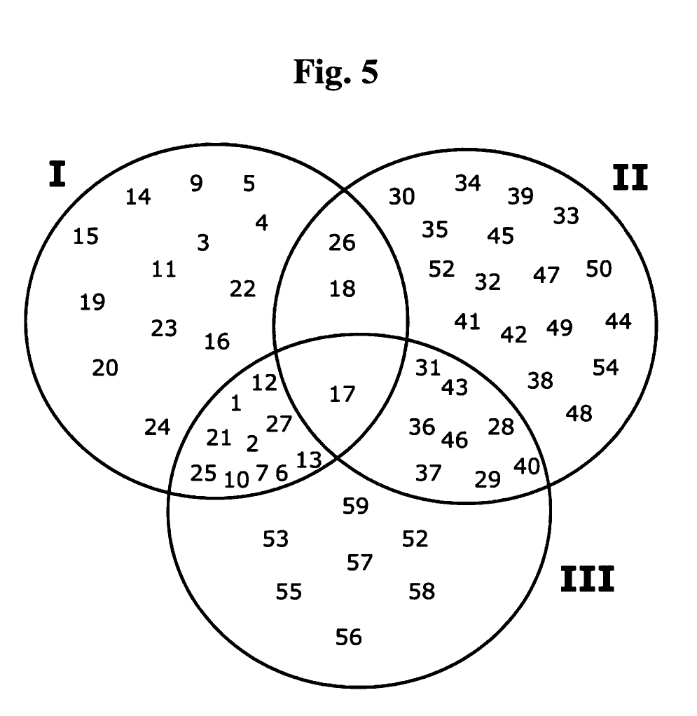

It can be seen that some of the antigens from the set of pre-CAD antigens (Table 2 List I) were also present in the post-CAD set (Table 2 List II), or in the set of antigens determined from the pre-CAD/post-CAD ratios (Table 2 List III). For example, three of the pre-CAD antigen reactivities were also prominent post-CAD (antigens 17, 18 and 26; Table 2 List I). The shared and distinct antigens are shown as a Venn diagram for the overlap between Antigen Lists I, II and III in FIG. 5. List III in Table 2 (ratio difference) can be seen to have generated a set of antigens most shared (indicated as H or S) between pre-CAD sera (List I) and post-CAD sera (List II); see Table 2 and FIG. 5.

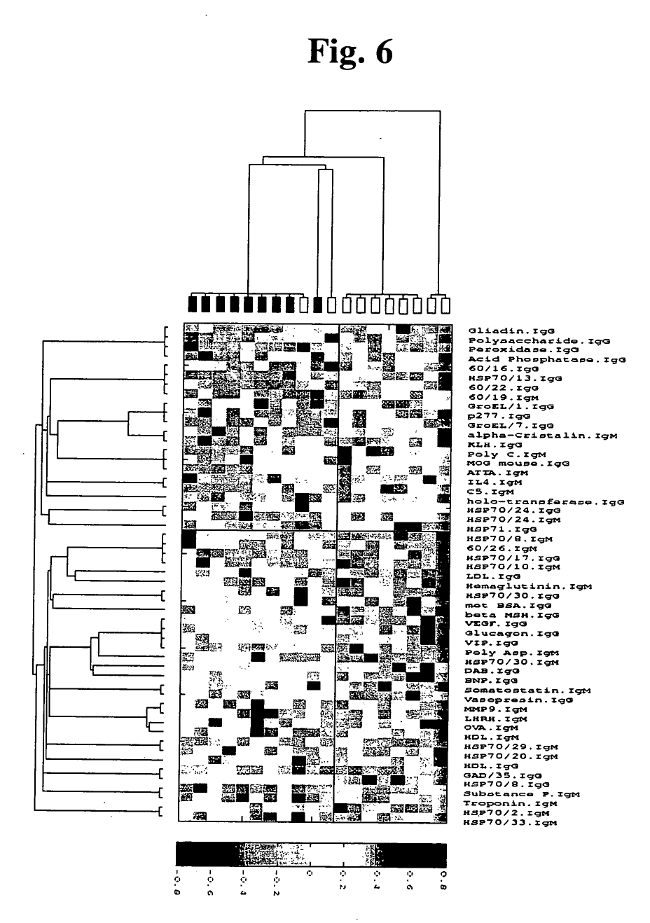

Informative IgM-reactive antigens were identified as described above for IgG-reactive antigens. A reactivity matrix depicting informative IgM-reactive antigens is shown in FIG. 6.

Tables 3 and 4, below, summarize the IgG- and IgM-reactive antigens diagnostic of type 1 diabetes or predisposition thereto, respectively.

TABLE 3
Summary of IgG-reactive antigens diagnostic of type 1 diabetes, or predisposition thereto

| Subject | | | |
|---|---|---|---|
| status* | Predictive antigens | Diagnostic antigens | Ratio A/B |
| SA | HEAT SHOCK PROTEIN | HEAT SHOCK PROTEIN | |
| | ANTIGENS - HUMAN, | ANTIGENS - HUMAN, | |
| | HSP70 | HSP60 | |
| | HSP70 peptide | HSP60 peptide | HSP60 peptide |
| | (SEQ ID NO: 24) | (SEQ ID NO: 19) | (SEQ ID NO: 14) |
| | HSP70 peptide | HEAT SHOCK PROTEIN | HSP60 peptide No. 18 |
| | (SEQ ID NO: 31) | ANTIGENS - HUMAN, | |
| | | HSP70 | |
| | HSP70 peptide | HSP70 peptide | HSP60 peptide No. 22 |
| | (SEQ ID NO: 37) | (SEQ ID NO: 22) | |
| | HORMONE ANTIGENS - | HSP70 peptide | HSP60 peptide No. 30 |
| | CARDIAC HORMONE | (SEQ ID NO: 23) | |
| | BNP | HSP70 peptide | HSP60 peptide |
| | | (SEQ ID NO: 25) | (SEQ ID NO: 19) |
| | HORMONE ANTIGENS - | HSP70 peptide | HSP70 peptide |
| | NEUROTRANSMITTER | (SEQ ID NO: 28) | (SEQ ID NO: 23) |
| | VIP | HSP70 peptide | HSP70 peptide |
| | | (SEQ ID NO: 33) | (SEQ ID NO: 29) |
| | HORMONE ANTIGENS - | IMMUNE SYSTEM | HSP70 peptide |
| | PIGMENTATION | ANTIGENS - | (SEQ ID NO: 33) |
| | HORMONE | CYTOKINE RECEPTOR | |
| | beta-MSH | IL-2R alpha-chain peptide | GAD peptide |
| | | (SEQ ID NO: 40) | (SEQ ID NO: 45) |
| | HORMONE ANTIGENS - | IL-2R beta-chain peptide | MOBP peptide |
| | VASCULAR HORMONE | (SEQ ID NO: 41) | (SEQ ID NO: 47) |
| | vasopressin | IMMUNE SYSTEM | cartilage extract |
| | | ANTIGENS - | |
| | | T-CELL RECEPTOR (TCR) | |
| | VEGF | TCR beta-chain peptide | TCR beta-chain peptide |
| | | (SEQ ID NO: 42) | (SEQ ID NO: 44) |
| | PLASMA ANTIGENS - | TCR) beta-chain peptide | holo-transferrin |
| | ALBUMIN | (SEQ ID NO: 44) | |
| | methylated BSA | METABOLIC ENZYME | human serum albumin |
| | | ANTIGENS - VARIOUS | |
| | PLASMA ANTIGENS - | aldolase | KLH |
| | LIPOPROTEIN | | |
| | HDL | GSTase | |
| | LDL | PLASMA ANTIGENS - | |
| | | COAGULATION | |
| | | REGULATOR | |
| | TISSUE ANTIGENS - | plasmin | |
| | NERVE SYSTEM | | |
| | MOLECULE | | |
| | GAD peptide | TISSUE ANTIGENS - | |
| | (SEQ ID NO: 46) | NERVE SYSTEM | |
| | | MOLECULE | |
| | TISSUE ANTIGENS - | GAD peptide | |
| | PANCREATIC | (SEQ ID NO: 45) | |
| | MOLECULE | | |
| | DAP | MOBP peptide | |
| | | (SEQ ID NO: 47) | |
| | glucagon | TISSUE ANTIGENS - | |
| | | PANCREATIC | |
| | | MOLECULE | |
| | | C-peptide | |
| HA | HEAT SHOCK PROTEIN | HEAT SHOCK PROTEIN | HSP60 peptide No. 35 |
| | ANTIGENS - BACTERIAL, | ANTIGENS - | |
| | E. COLI GROEL | BACTERIAL, E. COLI | |
| | | GROEL | |
| | GroEL peptide | GroEL peptide | GroEL peptide |
| | (SEQ ID NO: 1) | (SEQ ID NO: 1) | (SEQ ID NO: 5) |
| | GroEL peptide | GroEL peptide | GroEL peptide |
| | (SEQ ID NO: 3) | (SEQ ID NO: 4) | (SEQ ID NO: 8) |
| | HEAT SHOCK PROTEIN | GroEL peptide | GroEL peptide |
| | ANTIGENS - HUMAN, | (SEQ ID NO: 5) | (SEQ ID NO: 12) |
| | HSP60 | | |
| | HSP60 peptide | GroEL peptide | GroEL peptide No. 25 |
| | (SEQ ID NO: 14) | (SEQ ID NO: 7) | |
| | HSP60 peptide | GroEL peptide | GroEL peptide No. 28 |
| | (SEQ ID NO: 16) | (SEQ ID NO: 10) | |
| | HSP60 peptide | GroEL peptide | HSP70 peptide |
| | (SEQ ID NO: 20) | (SEQ ID NO: 12) | (SEQ ID NO: 24) |
| | HEAT SHOCK PROTEIN | HEAT SHOCK PROTEIN | glucagon |
| | ANTIGENS - HUMAN, | ANTIGENS - HUMAN, | |
| | HSP70 | HSP60 | |
| | HSP70 peptide | HSP60 peptide | rat TCR beta-chain C2 |
| | (SEQ ID NO: 29) | (SEQ ID NO: 20) | |
| | HSP70 peptide | HEAT SHOCK PROTEIN | vimentin |
| | (SEQ ID NO: 35) | ANTIGENS - HUMAN, | |
| | | HSP70 | |
| | HEAT SHOCK PROTEIN | HSP70 peptide | beta-MSH |
| | ANTIGENS - BACTERIAL, | (SEQ ID NO: 34) | |
| | M. TUBERCULOSIS | | |
| | HSP71 | METABOLIC ENZYME | somatostatin |
| | | ANTIGENS - VARIOUS | |
| | METABOLIC ENZYME | collagenase | vasopressin |
| | ANTIGENS - VARIOUS | | |
| | acid phosphatase | | VIP |
| | holo-transferrin | | methylated BSA |
| | HRP | | LDL |
| | MICROBIAL ANTIGENS - | | |
| | LIPOPOLYSACCHARIDE | | |
| | PS4 | | |
| | MOLLUSCAN ANTIGENS | | |
| | KLH | | |
| | PLANT ANTIGEN - | | |
| | WHEAT ANTIGEN | | |
| | gliadin | | |
| | TISSUE ANTIGENS - | | |
| | NERVE SYSTEM | | |
| | MOLECULE | | |
| | MOG | | |

*SA, test subject sick after disease-inductive treatment; HA, test subject healthy after disease-inductive treatment

TABLE 4
Summary of IgM-reactive antigens diagnostic of type 1 diabetes, or predisposition thereto

| Subject | | | |
|---|---|---|---|
| status* | Predictive antigens | Diagnostic antigens | Ratio A/B |
| SA | CELL/TISSUE | HEAT SHOCK PROTEIN | HSP60, full-length |
| | STRUCTURAL ANTIGENS - | ANTIGENS - HUMAN, | |
| | MUSCLE STRUCTURE | HSP70 | |
| | PROTEIN | | |
| | troponin | HSP70 peptide | HSP60 peptide No. 12 |
| | | (SEQ ID NO: 26) | |
| | HEAT SHOCK PROTEIN | HSP70 peptide | HSP70 peptide |
| | ANTIGENS - HUMAN, | (SEQ ID NO: 27) | (SEQ ID NO: 29) |
| | HSP70 | | |
| | HSP70 peptide | HSP70 peptide | cartilage extract |
| | (SEQ ID NO: 21) | (SEQ ID NO: 29) | |
| | HSP70 peptide | HSP70 peptide | dsDNA |
| | (SEQ ID NO: 24) | (SEQ ID NO: 30) | |
| | HSP70 peptide | HSP70 peptide | TNF-alpha |
| | (SEQ ID NO: 26) | (SEQ ID NO: 33) | |
| | HSP70 peptide | HSP70 peptide | ANP |
| | (SEQ ID NO: 32) | (SEQ ID NO: 38) | |
| | HSP70 peptide | IMMUNE SYSTEM | GST |
| | (SEQ ID NO: 36) | ANTIGENS - CYTOKINE | |
| | HSP70 peptide | TNF-alpha | C5 |
| | (SEQ ID NO: 37) | | |
| | HSP70 peptide | METABOLIC ENZYME | KLH |
| | (SEQ ID NO: 39) | ANTIGENS - VARIOUS | |
| | HOMOPOLYMERIC | GST | AT₁₈A |
| | POLYPEPTIDE ANTIGENS | | |
| | poly(Asp) | MICROBIAL ANTIGENS - | |
| | | VIRAL ANTIGEN | |
| | HORMONE ANTIGENS - | hemagglutinin | |
| | HYPOTHALAMIC | | |
| | HORMONE | | |
| | LHRH | MOLLUSCAN ANTIGENS | |
| | somatostatin | KLH | |
| | HORMONE ANTIGENS - | NUCLEIC ACID | |
| | NEUROTRANSMITTER | ANTIGENS | |
| | HORMONE | | |
| | substance P | dsDNA | |
| | METABOLIC ENZYME | ssDNA | |
| | ANTIGENS - MATRIX | | |
| | METALLOPROTEINASE | | |
| | (MMP) | | |
| | MMP9 | PLASMA ANTIGENS - | |
| | | ALBUMIN | |
| | MICROBIAL ANTIGENS - | OVA | |
| | VIRAL ANTIGEN | | |
| | hemagglutinin | PLASMA ANTIGENS - | |
| | | LIPOPROTEIN | |
| | PLANT ANTIGEN - | HDL | |
| | WHEAT ANTIGEN | | |
| | gliadin | TISSUE ANTIGENS - | |
| | | CARTILAGE ANTIGEN | |
| | PLASMA ANTIGENS - | cartilage extract | |
| | ALBUMIN | | |
| | OVA | TISSUE ANTIGENS - | |
| | | NERVE SYSTEM | |
| | | MOLECULE | |
| | PLASMA ANTIGENS - | synuclein | |
| | LIPOPROTEIN | | |
| | HDL | | |
| HA | HEAT SHOCK PROTEIN | HEAT SHOCK PROTEIN | GroEL peptide No. 28 |
| | ANTIGENS - | ANTIGENS - BACTERIAL, | |
| | CRYSTALLIN | E. COLI GROEL | |
| | alpha-crystallin | GroEL peptide | GroEL peptide No. 30 |
| | | (SEQ ID NO: 6) | |
| | HEAT SHOCK PROTEIN | GroEL peptide | GroEL peptide No. 33 |
| | ANTIGENS - BACTERIAL, | (SEQ ID NO: 7) | |
| | E. COLI GROEL | | |
| | GroEL peptide | GroEL peptide | GroEL peptide No. 34 |
| | (SEQ ID NO: 2) | (SEQ ID NO: 11) | |
| | GroEL peptide | HEAT SHOCK PROTEIN | Mt3 |
| | (SEQ ID NO: 4) | ANTIGENS - HUMAN, | |
| | | HSP60 | |
| | GroEL peptide | HSP60 peptide | HSP70 peptide |
| | (SEQ ID NO: 6) | (SEQ ID NO: 13) | (SEQ ID NO: 21) |
| | GroEL peptide | HSP60 peptide | collagen I |
| | (SEQ ID NO: 7) | (SEQ ID NO: 18) | |
| | GroEL peptide | HEAT SHOCK PROTEIN | collagen X |
| | (SEQ ID NO: 8) | ANTIGENS - HUMAN, | |
| | | HSP70 | |
| | GroEL peptide | HSP70 peptide | substance P |
| | (SEQ ID NO: 9) | (SEQ ID NO: 21) | |
| | HEAT SHOCK PROTEIN | IMMUNE SYSTEM | thyroglobulin |
| | ANTIGENS - HUMAN, | ANTIGENS - | |
| | HSP60 | T-CELL RECEPTOR (TCR) | |
| | HSP60 peptide | TCR beta-chain C2 | MMP9 |
| | (SEQ ID NO: 15) | | |
| | HSP60 peptide | MICROBIAL ANTIGENS - | |
| | (SEQ ID NO: 17) | LIPOPOLYSACCHARIDE | |
| | HEAT SHOCK PROTEIN | PS4 | |
| | ANTIGENS - HUMAN, | | |
| | HSP70 | | |
| | HSP70 peptide | TISSUE ANTIGENS - | |
| | (SEQ ID NO: 35) | PANCREATIC | |
| | | MOLECULE | |
| | IMMUNE SYSTEM | insulin chain B | |
| | ANTIGENS - | | |
| | COMPLEMENT | | |
| | MOLECULE | | |
| | C5 | | |
| | IMMUNE SYSTEM | | |
| | ANTIGENS - CYTOKINE | | |
| | IL-4 | | |
| | IL-10 | | |
| | NUCLEIC ACID | | |
| | AT₁₈A | | |
| | poly(C) | | |

*SA, test subject sick after disease-inductive treatment; HA, test subject healthy after disease-inductive treatment

Discussion: The presently disclosed antigen microarray required substantial preliminary work to obtain consistent results, including determination of a workable surface coating for the glass, reagent concentrations and incubation times, size of spots, distances between spots, washing protocols, laser activation and reading, and other technical issues.

In the presently disclosed experiments, it was surprisingly shown that the patterns of antigenic specificities of IgG antibodies displayed pre-CAD in male NOD mice can mark susceptibility or resistance to subsequently induced CAD. Patterns of antigenic specificities of IgG antibodies characteristic of healthy or diabetic mice post-CAD were also found, but these patterns required sets of antigens that differed from the informative pre-CAD set (see Table 2). Thus, IgG reactivities to some antigens may mark future susceptibility to CAD, but not CAD itself once the disease emerges, and, conversely, some IgG reactivities may mark the disease but not the susceptibility. Hence, prediction of future disease and diagnosis of present disease can depend on different data sets of information, at least in the CAD model. The reasons for this divergence need to be investigated, but the divergence itself may be explained by the likelihood that the IgG antibodies measured are not themselves the causal agents, but only indirect, surrogate markers for the autoimmune T-cells that directly regulate or mediate the diabetic process. This observation indicates the possibility of a similar divergence between the prediction and the diagnosis of human diseases.

Another notable finding was that health, both pre-CAD and post-CAD, was associated with relatively high IgG autoreactivity to self-antigens, to which the susceptible mice were low IgG responders (FIGS. 2 and 4, and Table 2). Thus some types of active autoimmunity may actually protect against autoimmune disease (Elias, D. et al., 1991. Proc Natl Acad Sci USA 88, 3088-91; Quintana, F. J. et al., 2002. J Immunol 169, 6030-5; Quintana, F. J. et al., 2002. J Immunol 169, 3422-8). This finding is compatible with the idea that autoimmunity of certain specificities is not only compatible with health, but essential for health [19; Cohen, I. R. (2000) Tending Adam's Garden: Evolving the Cognitive Immune Self (Academic Press, London)].

The cumulative experience of the immune system (including, for example, positive autoimmunity to antigens such as the lower 8 antigens in FIG. 2, Left Panel) can determine the organization of its component molecules and cells regarding self-antigens, and this internal structuring can, in turn, help one resist the accidental induction of an autoimmune disease. Type 1 diabetes appears in very young people (Tisch, R. & McDevitt, H., 1996. Cell 85, 291-7), so critical aspects of autoimmune organization must occur fairly early in one's lifetime. The results of this bioinformatic study would suggest that, in addition to individual differences in immune repertoires, some organized patterns of antigenic specificities of IgG autoantibodies are shared by groups of individuals, at least among NOD mice.

The presently disclosed bioinformatic analysis relates to two separate, but linked issues: predictive medicine via functional immunomics and the biological meaning of the autoimmune repertoire.

Functional immunomics may be defined as the functional state of the immune system inscribed in its global patterns of immune molecules and cells. Functional immunomics, even that limited to part of the IgG autoantibody repertoire as is presently disclosed, can help anticipate disease before it emerges, and anticipation is an important first step in predictive medicine.

Beyond its potential usefulness for predictive medicine, functional immunomics may be informative about the biology of immune system organization. Surprisingly the list of informative antigens (Table 2) does not contain insulin, a well-studied self-antigen in diabetes (Tisch, R. & McDevitt, H., 1996. Cell 85, 291-7). A peptide of glutamic acid decarboxylase (GAD), used clinically to diagnose type 1 diabetes in humans (Leslie, D. et al., 2001. J Clin Invest 108, 1417-22; Verge, C. F. et al., 1998. Diabetes 47, 1857-1866), was only informative after CAD was induced (Table 2 List II). The immune system is a complex system, and reactivities of seerningly minor magnitude can play major roles in complex system behavior (Auffray, C. et al., 2003. Philos Transact Ser A Math Phys Eng Sci. 361, 1125-39). Measuring autoantibodies to a few selected antigens only may not provide the same information as can a global pattern.

The presently disclosed experiments investigated patterns of antigenic specificities of antibodies, and not their function in the disease process. Nevertheless, the list of informative antigens is not unconnected to other observations regarding the pathophysiology type 1 diabetes. Six of the eight antigens to which relatively high IgG reactivity is associated with resistance to CAD are peptides derived from heat shock proteins (HSP): peptides p277, 22 and 16 of HSP60, peptides 1 and 7 from the sequence of GroEL (the “HSP60” molecule of E. coli), and peptide 13 of HSP70. Vaccination with HSP60/p277 can arrest type 1 diabetes in NOD mice (34), and has been shown to arrest the destruction of insulin-producing beta-cells in a clinical trial in humans (Raz, I. et al., 2001. The Lancet 358, 1749-1753).

It is worthy of note that peptide p277 of HSP60 was first discovered as a dominant epitope for T-cells (Elias, D. et al., 1991. Proc Natl Acad Sci USA 88, 3088-91). The natural IgG reactivity of CAD resistant mice to this “T-cell peptide” demonstrates that some autoantibodies do reflect elements of the T-cell repertoire. Indeed, prevention of spontaneous diabetes in NOD mice by stimulating innate toll-like receptors with CpG oligonucleotide was found to spontaneously activate the production of IgG antibodies to peptide p277 (Quintana, F. J. et al., 2000. J. Immunol. 165, 6148-55). It has been found that vaccination with HSP60 can inhibit CAD, apparently by modifying the cytokine profile of autoimmune effector T-cells (Quintana, F. J. et al., 2002. J Immunol 169, 6030-5). HSP60 vaccination can also induce regulatory T-cells effective in models of autoimmune arthritis (Quintana, F. J. et al., 2002. J Immunol 169, 3422-8; Quintana, F. J. et al., 2003. J Immunol 171, 3533-41). Thus the association of resistance to CAD with natural IgG antibodies to HSP60 peptides suggests that medicinal vaccination with HSP60 or its peptides may work by strengthening regulatory networks that arise naturally through immune experience with endogenous (or cross-reactive bacterial) heat shock proteins. Autoimmunity to HSP60 peptides like p277 is built into the healthy immune system.

The 19 antigens targeted by IgG antibodies in the CAD-susceptible mice are also interesting biologically. Three peptides of HSP70 are included, and T-cell autoimmunity to HSP70 has been described in human type 1 diabetes patients (Abulafia-Lapid, R. et al., 2003. J Autoimmun 20, 313-21). Gliadin is an antigen associated with celiac disease, and celiac patients have been reported to have an increased incidence of type 1 diabetes (Schuppan, D. & Hahn, E. G., 2001. J Pediatr Endocrinol Metab 14, 597-605.). MOG is a molecule present in myelin, and T-cell autoimmunity to MOG can induce experimental autoimmune encephalomyelitis in NOD mice (Slavin, A. et al., 1998. Autoimmunity 28, 109-20). Glucagon is produced by alpha-cells in the pancreatic islets adjacent to the beta-cells that produce insulin, but no studies of autoimmunity glucagon have been yet reported in type 1 diabetes. Accelerated atherosclerosis is a serious complication of type 1 diabetes (Frostegard, J., 2002. Autoimmun Rev 1, 233-7), and is assumed to arise as a complication of poor glucose homeostasis in poorly controlled diabetes (Jensen-Urstad, K. J. et al., 1996. Diabetes 45, 1253-8). Autoimmunity to LDL and HDL, however, has been proposed to be a factor in atherosclerosis in general (Frostegard, J., 2002. Autoimmun Rev 1, 233-7). The finding of heightened IgG autoimmunity to LDL and HDL in the mice susceptible to CAD suggests the possibility that LDL and HDL autoimmunity might actually be part of the collective of autoimmune reactions responsible for the primary development of type 1 diabetes. If this is true, then the vascular “complications” of type 1 diabetes may be a primary and early event in the disease process and not merely a phenomenon secondary to poor metabolic control. VEGF and vasopressin too are molecules that function in blood vessel formation and the physiology of blood flow (Mor, F. et al., 2004. J Immunol 172, 4618-23; Neufeld, G. et al., 1999. Faseb J 13, 9-22). The increase in IgG antibodies to certain antigens post-CAD is also intriguing. For now, it is important to note that a bioinformatic analysis can, by itself, raise new questions for further biological research; arrays of antigens open new windows for viewing natural autoimmunity, autoimmune disease and the links between them.

The demonstration of patterns of autoantibody reactivities with key self-molecules and the association of such reactivities with health challenges basic assumptions of the classical clonal selection theory (CST) of adaptive immunity [Burnet, F. M. (1959) The clonal selection theory of acquired immunity (Cambridge University Press)]. According to the CST, autoimmune repertoires should not exist in healthy individuals. The present findings are more compatible with a cognitive paradigm of immunity [Cohen, I. R. (2000) Tending Adam's Garden: Evolving the Cognitive Immune Self (Academic Press, London); Cohen, I. R. and Young, D. B., 1991. Immunol Today 12, 105-10; Cohen, I. R., 1992. Immunol Today 13, 490-4).

The core of organized autoimmune repertoires within the immune system has been termed the immunological homunculus, the immune system's internal representation of the body under its care (Cohen, I. R. & Young, D. B., 1991. Immunol Today 12, 105-10; Cohen, I. R., 1992. Immunol Today 13, 490-4). The mammalian immune system, in addition to its well-studied role in defending the body against foreign invaders, is now understood to be heavily involved in maintaining the integrity of the body from within; immune system cells and molecules, which comprise the inflammatory response, are key factors in wound healing, neuroprotection, connective tissue formation, angiogenesis, tissue morphology and regeneration, and waste disposal [Lacroix-Desmazes, S. et al., 1998. J Immunol Methods 216, 117-37; Schwartz, M. & Cohen, I. R., 2000. Immunol Today 21, 265-8; Cohen, I. R. (2000) Tending Adam's Garden: Evolving the Cognitive Immune Self (Academic Press, London)]. To dispense reparative inflammation at the right sites and occasions, the immune system has to assess the state of the body on the fly. In this respect, the immune system acts as it were the body's onboard bioinformatic computer. If so, predictive medicine would do well to mine this immune information, as presently disclosed study suggests it might.

Summary: One's present repertoire of antibodies encodes the history of one's past immunological experience. The presently disclosed studies addressed whether a global antibody antigenic specificity repertoire can be consulted to predict resistance or susceptibility to the future development of an immune disease. To address this issue, an antigen microarray chip was developed and bioinformatic analysis was used to study a model of type 1 diabetes developing in non-obese diabetic (NOD) male mice in which the disease was accelerated and synchronized by exposing the mice to cyclophosphamide at 4-weeks of age. Sera from 19 individual mice was obtained, cyclophosphamide-accelerated diabetes (CAD) was induced in the mice, and, characteristically for mice from the source colony employed, 9 mice became severely diabetic while 10 mice permanently resisted diabetes. Serum was again obtained from each mouse following CAD induction, and the patterns of antigenic specificities of serum antibodies in the individual mice to 266 different antigens spotted on the antigen chip were analyzed. A select panel of 27 different antigens (10 percent of the array) was identified, revealing a pattern of IgG antibody reactivity in the pre-CAD sera that discriminated between the mice resistant or susceptible to CAD with 100 percent sensitivity and 82 percent specificity (p=0.017). Surprisingly, the set of IgG antibodies that was informative prior to CAD induction did not separate the resistant and susceptible groups following the onset of CAD; new antigens became critical for post-CAD repertoire discrimination. Thus, at least for a model disease, present antibody repertoires can predict future disease; predictive and diagnostic repertoires can differ; and decisive information about immune system behavior can be mined by bioinformatic technology. Repertoires matter.

Conclusion: Novel antigen microarrays were designed and used to identify unexpected global patterns of antigenic specificities of IgG and IgM antibodies characteristic of type 1 diabetes, or predisposition thereto. As such, the presently described antigen probe sets, microarrays, and methods of their use enable improved diagnosis of type 1 diabetes, and predisposition thereto, relative to the prior art.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, and patent applications mentioned in this specification are herein incorporated in their entirety by reference into the specification, to the same extent as if each individual publication, patent, or patent application was specifically and individually indicated to be incorporated herein by reference. In addition, citation or identification of any reference in this application shall not be construed as an admission that such reference is available as prior art to the present invention.

1. A method of diagnosing an immune disease, or a predisposition thereto, in a subject, the method comprising determining a capacity of immunoglobulins of the subject to specifically bind each antigen probe of an antigen probe set, wherein said antigen probe set comprises a plurality of antigen probes selected from the group consisting of at least a portion of a cell/tissue structure molecule, at least a portion of a heat shock protein, at least a portion of an immune system molecule, at least a portion of a homopolymeric polypeptide, at least a portion of a hormone, at least a portion of a metabolic enzyme, at least a portion of a microbial antigen, at least a portion of a molluscan antigen, at least a portion of a nucleic acid, at least a portion of a plant antigen, at least a portion of plasma molecule, and at least a portion of a tissue antigen, wherein said capacity is indicative of the immune disease or the predisposition thereto, thereby diagnosing the immune disease, or the predisposition thereto, in the subject.

2. The method of claim 1, wherein said plurality of antigen probes comprises a number of distinct antigen probes selected from the group consisting of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, and 91 antigen probes.

3. The method of claim 1, wherein said immunoglobulins belong to the IgG isotype, and whereas said plurality of antigen probes is selected from the group consisting of at least a portion of a human 70 kDa heat shock protein, at least a portion of a cardiac hormone, at least a portion of a neurotransmitter, at least a portion of a pigmentation hormone, at least a portion of a vascular hormone, at least a portion of an albumin, at least a portion of a lipoprotein, at least a portion of a nerve system molecule, and at least a portion of a pancreatic molecule.

4. The method of claim 1, wherein said immunoglobulins belong to the IgG isotype, and whereas said plurality of antigen probes is selected from the group consisting of a bacterial heat shock protein, at least a portion of a 60 kDa human heat shock protein, at least a portion of a 70 kDa human heat shock protein, at least a portion of a metabolic enzyme, at least a portion of a lipopolysaccharide, at least a portion of a molluscan antigen, at least a portion of a wheat antigen, and at least a portion of a nerve system molecule.

5. The method of claim 1, wherein said immunoglobulins belong to the IgG isotype, and whereas said plurality of antigen probes is selected from the group consisting of at least a portion of a 60 kDa human heat shock protein, at least a portion of a 70 kDa human heat shock protein, at least a portion of a cytokine receptor, at least a portion of a T-cell receptor, at least a portion of a metabolic enzyme, at least a portion of a coagulation regulator, at least a portion of a nerve system molecule, and at least a portion of a pancreatic molecule.

6. The method of claim 1, wherein said immunoglobulins belong to the IgG isotype, and whereas said plurality of antigen probes is selected from the group consisting of at least a portion of a bacterial heat shock protein, at least a portion of a human 60 kDa heat shock protein, at least a portion of a human 70 kDa heat shock protein, and at least a portion of a glutathione S-transferase.

7. The method of claim 1, wherein said immunoglobulins belong to the IgM isotype, and whereas said plurality of antigen probes is selected from the group consisting of at least a portion of a muscle structure protein, at least a portion of a 70 kDa human heat shock protein, at least a portion of a homopolymeric polypeptide, at least a portion of a hypothalamic hormone, at least a portion of a neurotransmitter, at least a portion of a matrix metalloproteinase, at least a portion of a viral antigen, at least a portion of a wheat antigen, at least a portion of an albumin, and at least a portion of a lipoprotein.

8. The method of claim 1, wherein said immunoglobulins belong to the IgM isotype, and whereas said plurality of antigen probes is selected from the group consisting of at least a portion of a crystallin, at least a portion of a bacterial heat shock protein, at least a portion of a human 60 kDa heat shock protein, at least a portion of a human 70 kDa heat shock protein, at least a portion of a complement molecule, at least a portion of a cytokine, and at least a portion of a nucleic acid.

9. The method of claim 1, wherein said immunoglobulins belong to the IgM isotype, and whereas said plurality of antigen probes is selected from the group consisting of at least a portion of a human 70 kDa heat shock protein, at least a portion of a cytokine, at least a portion of a metabolic enzyme, at least a portion of a viral antigen, at least a portion of a molluscan antigen, at least a portion of an albumin, at least a portion of a lipoprotein, at least a portion of a cartilage antigen, and at least a portion of a nerve system molecule.

10. The method of claim 1, wherein said immunoglobulins belong to the IgM isotype, and whereas said plurality of antigen probes is selected from the group consisting of at least a portion of a bacterial heat shock protein, at least a portion of a human 60 kDa heat shock protein, at least a portion of a human 70 kDa heat shock protein, at least a portion of T-cell receptor, at least a portion of a lipopolysaccharide and at least a portion of a pancreatic molecule.

11. The method of claim 1, wherein said determining said capacity of said immunoglobulins to specifically bind each antigen probe of said plurality of antigen probes comprises:
(a) exposing said immunoglobulins to said plurality of antigen probes, wherein each antigen probe of said plurality of antigen probes is attached to a distinct addressable location of a plurality of addressable locations of a support of an antigen probe array; and
(b) measuring a hybridization of said each antigen probe of said plurality of antigen probes with said immunoglobulins.

12. The method of claim 1, wherein said immune disease is an autoimmune disease.

13. The method of claim 1, wherein said immune disease is type I diabetes.

14. The method of claim 1, wherein said immune disease is a pancreatic immune disease.

15. An antigen probe array comprising:
(a) a support which comprises a plurality of addressable locations; and
(b) an antigen probe set which comprises a plurality of antigen probes selected from the group consisting of at least a portion of a cell/tissue structure molecule, at least a portion of a heat shock protein, at least a portion of an immune system molecule, at least a portion of a homopolymeric polypeptide, at least a portion of a hormone, at least a portion of a metabolic enzyme, at least a portion of a microbial antigen, at least a portion of a molluscan antigen, at least a portion of a nucleic acid, at least a portion of a plant antigen, at least a portion of plasma molecule, and at least a portion of a tissue antigen, wherein each antigen probe of said plurality of antigen probes is attached to a specific addressable location of said plurality of addressable locations.

16. The antigen probe array of claim 15, wherein said plurality of antigen probes comprises a number of distinct antigen probes selected from the group consisting of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, and 91 antigen probes.

17. The antigen probe array of claim 15, wherein said plurality of antigen probes is selected from the group consisting of at least a portion of a human 70 kDa heat shock protein, at least a portion of a cardiac hormone, at least a portion of a neurotransmitter, at least a portion of a pigmentation hormone, at least a portion of a vascular hormone, at least a portion of an albumin, at least a portion of a lipoprotein, at least a portion of a nerve system molecule, and at least a portion of a pancreatic molecule.

18. The antigen probe array of claim 15, wherein said plurality of antigen probes is selected from the group consisting of a bacterial heat shock protein, at least a portion of a 60 kDa human heat shock protein, at least a portion of a 70 kDa human heat shock protein, at least a portion of a metabolic enzyme, at least a portion of a lipopolysaccharide, at least a portion of a molluscan antigen, at least a portion of a wheat antigen, and at least a portion of a nerve system molecule.

19. The antigen probe array of claim 15, wherein said plurality of antigen probes is selected from the group consisting of at least a portion of a 60 kDa human heat shock protein, at least a portion of a 70 kDa human heat shock protein, at least a portion of a cytokine receptor, at least a portion of a T-cell receptor, at least a portion of a metabolic enzyme, at least a portion of a coagulation regulator, at least a portion of a nerve system molecule, and at least a portion of a pancreatic molecule.

20. The antigen probe array of claim 15, wherein said plurality of antigen probes is selected from the group consisting of at least a portion of a bacterial heat shock protein, at least a portion of a human 60 kDa heat shock protein, at least a portion of a human 70 kDa heat shock protein, and at least a portion of a glutathione S-transferase.

21. The antigen probe array of claim 15, wherein said plurality of antigen probes is selected from the group consisting of at least a portion of a muscle structure protein, at least a portion of a 70 kDa human heat shock protein, at least a portion of a homopolymeric polypeptide, at least a portion of a hypothalamic hormone, at least a portion of a neurotransmitter, at least a portion of a matrix metalloproteinase, at least a portion of a viral antigen, at least a portion of a wheat antigen, at least a portion of an albumin, and at least a portion of a lipoprotein.

22. The antigen probe array of claim 15, wherein said plurality of antigen probes is selected from the group consisting of at least a portion of a crystallin, at least a portion of a bacterial heat shock protein, at least a portion of a human 60 kDa heat shock protein, at least a portion of a human 70 kDa heat shock protein, at least a portion of a complement molecule, at least a portion of a cytokine, and at least a portion of a nucleic acid.

23. The antigen probe array of claim 15, wherein said plurality of antigen probes is selected from the group consisting of at least a portion of a human 70 kDa heat shock protein, at least a portion of a cytokine, at least a portion of a metabolic enzyme, at least a portion of a viral antigen, at least a portion of a molluscan antigen, at least a portion of an albumin, at least a portion of a lipoprotein, at least a portion of a cartilage antigen, and at least a portion of a nerve system molecule.

24. The antigen probe array of claim 15, wherein said plurality of antigen probes is selected from the group consisting of at least a portion of a bacterial heat shock protein, at least a portion of a human 60 kDa heat shock protein, at least a portion of a human 70 kDa heat shock protein, at least a portion of T-cell receptor, at least a portion of a lipopolysaccharide and at least a portion of a pancreatic molecule.

25. An antigen probe set comprising a plurality of antigen probes selected from the group consisting of at least a portion of a cell/tissue structure molecule, at least a portion of a heat shock protein, at least a portion of an immune system molecule, at least a portion of a homopolymeric polypeptide, at least a portion of a hormone, at least a portion of a metabolic enzyme, at least a portion of a microbial antigen, at least a portion of a molluscan antigen, at least a portion of a nucleic acid, at least a portion of a plant antigen, at least a portion of plasma molecule, and at least a portion of a tissue antigen.

26. The antigen probe set of claim 25, wherein said plurality of antigen probes is selected from the group consisting of at least a portion of a human 70 kDa heat shock protein, at least a portion of a cardiac hormone, at least a portion of a neurotransmitter, at least a portion of a pigmentation hormone, at least a portion of a vascular hormone, at least a portion of an albumin, at least a portion of a lipoprotein, at least a portion of a nerve system molecule, and at least a portion of a pancreatic molecule.

27. The antigen probe set of claim 25, wherein said plurality of antigen probes is selected from the group consisting of a bacterial heat shock protein, at least a portion of a 60 kDa human heat shock protein, at least a portion of a 70 kDa human heat shock protein, at least a portion of a metabolic enzyme, at least a portion of a lipopolysaccharide, at least a portion of a molluscan antigen, at least a portion of a wheat antigen, and at least a portion of a nerve system molecule.

28. The antigen probe set of claim 25, wherein said plurality of antigen probes is selected from the group consisting of at least a portion of a 60 kDa human heat shock protein, at least a portion of a 70 kDa human heat shock protein, at least a portion of a cytokine receptor, at least a portion of a T-cell receptor, at least a portion of a metabolic enzyme, at least a portion of a coagulation regulator, at least a portion of a nerve system molecule, and at least a portion of a pancreatic molecule.

29. The antigen probe set of claim 25, wherein said plurality of antigen probes is selected from the group consisting of at least a portion of a bacterial heat shock protein, at least a portion of a human 60 kDa heat shock protein, at least a portion of a human 70 kDa heat shock protein, and at least a portion of a glutathione S-transferase.

30. The antigen probe set of claim 25, wherein said plurality of antigen probes is selected from the group consisting of at least a portion of a muscle structure protein, at least a portion of a 70 kDa human heat shock protein, at least a portion of a homopolymeric polypeptide, at least a portion of a hypothalamic hormone, at least a portion of a neurotransmitter, at least a portion of a matrix metalloproteinase, at least a portion of a viral antigen, at least a portion of a wheat antigen, at least a portion of an albumin, and at least a portion of a lipoprotein.

31. The antigen probe set of claim 25, wherein said plurality of antigen probes is selected from the group consisting of at least a portion of a crystallin, at least a portion of a bacterial heat shock protein, at least a portion of a human 60 kDa heat shock protein, at least a portion of a human 70 kDa heat shock protein, at least a portion of a complement molecule, at least a portion of a cytokine, and at least a portion of a nucleic acid.

32. The antigen probe set of claim 25, wherein said plurality of antigen probes is selected from the group consisting of at least a portion of a human 70 kDa heat shock protein, at least a portion of a cytokine, at least a portion of a metabolic enzyme, at least a portion of a viral antigen, at least a portion of a molluscan antigen, at least a portion of an albumin, at least a portion of a lipoprotein, at least a portion of a cartilage antigen, and at least a portion of a nerve system molecule.

33. The antigen probe set of claim 25, wherein said plurality of antigen probes is selected from the group consisting of at least a portion of a bacterial heat shock protein, at least a portion of a human 60 kDa heat shock protein, at least a portion of a human 70 kDa heat shock protein, at least a portion of T-cell receptor, at least a portion of a lipopolysaccharide and at least a portion of a pancreatic molecule.


SEQUENCE LISTING (47 sequences)

SEQ ID NO: 1 - protein, 20 aa, Artificial sequence
  Synthetic peptide
AAKDVKFGNDARVKMLRGVN

SEQ ID NO: 2 - protein, 20 aa, Artificial sequence
  Synthetic peptide
GPKGRNVVLDKSFGAPTITK

SEQ ID NO: 3 - protein, 20 aa, Artificial sequence
  Synthetic peptide
ATVLAQAIITEGLKAVAAGM

SEQ ID NO: 4 - protein, 20 aa, Artificial sequence
  Synthetic peptide
PCSDSKAIAQVGTISANSDE

SEQ ID NO: 5 - protein, 20 aa, Artificial sequence
  Synthetic peptide
ANSDETVGKLIAEAMDKVGK

SEQ ID NO: 6 - protein, 20 aa, Artificial sequence
  Synthetic peptide
RGYLSPYFINKPETGAVELE

SEQ ID NO: 7 - protein, 20 aa, Artificial sequence
  Synthetic peptide
AVELESPFILLADKKISNIR

SEQ ID NO: 8 - protein, 20 aa, Artificial sequence
  Synthetic peptide
ISNIREMLPVLEAVAKAGKP

SEQ ID NO: 9 - protein, 20 aa, Artificial sequence
  Synthetic peptide
KAGKPLLIIAEDVEGEALAT

SEQ ID NO: 10 - protein, 20 aa, Artificial sequence
  Synthetic peptide
EALATAVVNTIRGIVKVAAV

SEQ ID NO: 11 - protein, 20 aa, Artificial sequence
  Synthetic peptide
LGQAKRVVINKDTTTIIDGV

SEQ ID NO: 12 - protein, 20 aa, Artificial sequence
  Synthetic peptide
IIDGVGEEAAIQGRVAQIRQ

SEQ ID NO: 13 - protein, 20 aa, Artificial sequence
  Synthetic peptide
RKGVITVKDGKTLNDELEII

SEQ ID NO: 14 - protein, 20 aa, Artificial sequence
  Synthetic peptide
SPYFINTSKGQKCEFQDAYV

SEQ ID NO: 15 - protein, 20 aa, Artificial sequence
  Synthetic peptide
LVIIAEDVDGEALSTLVLNR

SEQ ID NO: 16 - protein, 20 aa, Artificial sequence
  Synthetic peptide
MAIATGGAVFGEEGLTLNLE

SEQ ID NO: 17 - protein, 20 aa, Artificial sequence
  Synthetic peptide
EQLDVTTSEYEKEKLNERLA

SEQ ID NO: 18 - protein, 20 aa, Artificial sequence
  Synthetic peptide
EIIKRTLKIPAMTIAKNAGV

SEQ ID NO: 19 - protein, 20 aa, Artificial sequence
  Synthetic peptide
QSSSEVGYDAMAGDFVNMVE

SEQ ID NO: 20 - protein, 24 aa, Artificial sequence
  Synthetic peptide
VLGGGVALLRVIPALDSLTPANED

SEQ ID NO: 21 - protein, 20 aa, Artificial sequence
  Synthetic peptide
SCVGVFQHGKVEIIANDQGN

SEQ ID NO: 22 - protein, 20 aa, Artificial sequence
  Synthetic peptide
DTERLIGDAAKNQVALNPQN

SEQ ID NO: 23 - protein, 20 aa, Artificial sequence
  Synthetic peptide
RKFGDPVVQSDMKHWPFQVI

SEQ ID NO: 24 - protein, 20 aa, Artificial sequence
  Synthetic peptide
SYKGETKAFYPEEISSMVLT

SEQ ID NO: 25 - protein, 20 aa, Artificial sequence
  Synthetic peptide
SMVLTKMKEIAEAYLGYPVT

SEQ ID NO: 26 - protein, 20 aa, Artificial sequence
  Synthetic peptide
GYPVTNAVITVPAYFNDSQR

SEQ ID NO: 27 - protein, 20 aa, Artificial sequence
  Synthetic peptide
NDSQRQATKDAGVIAGLNVL

SEQ ID NO: 28 - protein, 20 aa, Artificial sequence
  Synthetic peptide
GLNVLRIINEPTAAAIAYGL

SEQ ID NO: 29 - protein, 20 aa, Artificial sequence
  Synthetic peptide
IAYGLDRTGKGERNVLIFDL

SEQ ID NO: 30 - protein, 20 aa, Artificial sequence
  Synthetic peptide
LIFDLGGGTFDVSILTIDDG

SEQ ID NO: 31 - protein, 20 aa, Artificial sequence
  Synthetic peptide
FVEEFKRKHKKDISQNKRAV

SEQ ID NO: 32 - protein, 20 aa, Artificial sequence
  Synthetic peptide
SLFEGIDFYTSITRARFEEL

SEQ ID NO: 33 - protein, 20 aa, Artificial sequence
  Synthetic peptide
PVEKALRDAKLDKAQIHDLV

SEQ ID NO: 34 - protein, 20 aa, Artificial sequence
  Synthetic peptide
IHDLVLVGGSTRIPKVQKLL

SEQ ID NO: 35 - protein, 20 aa, Artificial sequence
  Synthetic peptide
VQKLLQDFFNGRDLNKSINP

SEQ ID NO: 36 - protein, 20 aa, Artificial sequence
  Synthetic peptide
PTKQTQIFTTYSDNQPGVLI

SEQ ID NO: 37 - protein, 20 aa, Artificial sequence
  Synthetic peptide
PGVLIQVYEGERAMTKDNNL

SEQ ID NO: 38 - protein, 20 aa, Artificial sequence
  Synthetic peptide
PAPGVPQIEVTFDIDANGIL

SEQ ID NO: 39 - protein, 20 aa, Artificial sequence
  Synthetic peptide
ANGILNVTATDKSTGKANKI

SEQ ID NO: 40 - protein, 20 aa, Artificial sequence
  Synthetic peptide
ASEESQGSRNSFPESEACPT

SEQ ID NO: 41 - protein, 21 aa, Artificial sequence
  Synthetic peptide
TTDTQKSTQSVYQENLAGHCR

SEQ ID NO: 42 - protein, 17 aa, Artificial sequence
  Synthetic peptide
DSPKPVTQNISAEAWGR

SEQ ID NO: 43 - protein, 17 aa, Artificial sequence
  Synthetic peptide
VLVSTLVVMTMVKRKSS

SEQ ID NO: 44 - protein, 17 aa, Artificial sequence
  Synthetic peptide
VTPPKVSLFEPSEAEIA

SEQ ID NO: 45 - protein, 20 aa, Artificial sequence
  Synthetic peptide
IPPSLRTLEDNEERMSRLSK

SEQ ID NO: 46 - protein, 20 aa, Artificial sequence
  Synthetic peptide
SRLSKVAPVIKARMMEYGTT

SEQ ID NO: 47 - protein, 13 aa, Artificial sequence
  Synthetic peptide
RPKQQPAAPPAVV